SEQUENCE LISTING (9 sequences)

SEQ ID NO: 1 - protein, 1037 aa, Artificial Sequence
  Description of Artificial Sequence Synthetic polypeptide
MAVIIFVNGIRAVNGLVKSSINTANAFAEEGLDVHLINFVGNITGAEHLYPPFHLHPNVKTSSIIDLFNDIPENVSCRNTPFYSIHQQFFKAEYSAHYKHVLMKIESLLSAEDSIIFTHPLQLEMYRLANNDIKSKAKLIVQIHGNYMEEIHNYEILARNIDYVDYLQTVSDEMLEEMHSHFKIKKDKLVFIPNITYPISLEKKEADFFIKDNEDIDNAQKFKRISIVGSIQPRKNQLDAIKIINKIKNENYILQIYGKSINKDYFELIKKYIKDNKLQNRILFKGESSEQEIYENTDILIMTSESEGFPYIFMEGMVYDIPIVVYDFKYGANDYSNYNENGCVFKTGDISGMAKKIIELLNNPEKYKELVQYNHNRFLKEYAKDVVMAKYFTILPRSFNNVSLSSAFSRKELDEFQNITFSIEDSNDLAHIWNFELTNPAQNMNFFALVGKRKFPMDAHIQGTQCTIKIAHKKTGNLLSLLLKKRNQLNLSRGYTLIAEDNSYEKYIGAISNKGNFEIIANKKSSLVTINKSTLELHEIPHELHQNKLLIALPNMQTPLKITDDNLIPIQASIKLEKIGNTYYPCFLPSGIFNNICLDYGEESKIINFSKYSYKYIYDSIRHIEQHTDISDIIVCNVYSWELIRASVIESLMEFTGKWEKHFQTSPKIDYRFDHEGKRSMDDVFSEETFIMEFPRKNGIDKKTAAFQNIPNSIVMEYPQTNGYSMRSHSLKSNVVAAKHFLEKLNKIKVDIKFKKHDLANIKKMNRIIYEHLGININIEAFLKPRLEKFKREEKYFHDFFKRNNFKEVIFPSTYWNPGIICAAHKQGIKVSDIQYAAITPYHPAYFKSPKSHYVADKLFLWSEYWNHELLPNPTREIGSGAAYWYALDDVRFSEKLNYDYIFLSQSRISSRLLSFAIEFALKNPQLQLLFSKHPDENIDLKNRIIPDNLIISTESSIQGINESRVAVGVYSTSLFEALACGKQTFVVKYPGYEIMSNEIDSGLFFAVETPEEMLEKTSPNWVAVADIENQFFGQEK

SEQ ID NO: 2 - protein, 1045 aa, Artificial Sequence
  Description of Artificial Sequence Synthetic polypeptide
MAVIIFVNGIRAVNGLVKSSINTANAFAEEGLDVHLINFVGNITGAEHLYPPFHLHPNVKTSSIIDLFNDIPENVSCRNTPFYSIHQQFFKAEYSAHYKHVLMKIESLLSAEDSIIFTHPLQLEMYRLANNDIKSKAKLIVQIHGNYMEEIHNYEILARNIDYVDYLQTVSDEMLEEMHSHFKIKKDKLVFIPNITYPISLEKKEADFFIKDNEDIDNAQKFKRISIVGSIQPRKNQLDAIKIINKIKNENYILQIYGKSINKDYFELIKKYIKDNKLQNRILFKGESSEQEIYENTDILIMTSESEGFPYIFMEGMVYDIPIVVYDFKYGANDYSNYNENGCVFKTGDISGMAKKIIELLNNPEKYKELVQYNHNRFLKEYAKDVVMAKYFTILPRSFNNVSLSSAFSRKELDEFQNITFSIEDSNDLAHIWNFELTNPAQNMNFFALVGKRKFPMDAHIQGTQCTIKIAHKKTGNLLSLLLKKRNQLNLSRGYTLIAEDNSYEKYIGAISNKGNFEIIANKKSSLVTINKSTLELHEIPHELHQNKLLIALPNMQTPLKITDDNLIPIQASIKLEKIGNTYYPCFLPSGIFNNICLDYGEESKIINFSKYSYKYIYDSIRHIEQHTDISDIIVCNVYSWELIRASVIESLMEFTGKWEKHFQTSPKIDYRFDHEGKRSMDDVFSEETFIMEFPRKNGIDKKTAAFQNIPNSIVMEYPQTNGYSMRSHSLKSNVVAAKHFLEKLNKIKVDIKFKKHDLANIKKMNRIIYEHLGININIEAFLKPRLEKFKREEKYFHDFFKRNNFKEVIFPSTYWNPGIICAAHKQGIKVSDIQYAAITPYHPAYFKSPKSHYVADKLFLWSEYWNHELLPNPTREIGSGAAYWYALDDVRFSEKLNYDYIFLSQSRISSRLLSFAIEFALKNPQLQLLFSKHPDENIDLKNRIIPDNLIISTESSIQGINESRVAVGVYSTSLFEALACGKQTFVVKYPGYEIMSNEIDSGLFFAVETPEEMLEKTSPNWVAVADIENQFFGQEKLEHHHHHH

SEQ ID NO: 3 - protein, 228 aa, Artificial Sequence
  Description of Artificial Sequence Synthetic polypeptide
MEKQNIAVILARQNSKGLPLKNLRKMNGISLLGHTINAAISSKCFDRIIVSTDGGLIAEEAKNFGVEVVLRPAELASDTASSISGVIHALETIGSNSGTVTLLQPTSPLRTGAHIREAFSLFDEKIKGSVVSACPMEHHPLKTLLQINNGEYAPMRHLSDLEQPRQQLPQAFRPNGAIYINDTASLIANNCFFIAPTKLYIMSHQDSIDIDTELDLQQAENILNHKES

SEQ ID NO: 4 - protein, 509 aa, Artificial Sequence
  Description of Artificial Sequence Synthetic polypeptide
MTEINDKAQLDIAAAGDTDAVTSDTPEETVNTPEVDETFELSAAKMREHGMSETAINQFHHLYDVWRHEEASSWIREDDIEPLGHVPSFHDVYETINHDKAVDAFAKTAFLKLNGGLGTSMGLDKAKSLLPVRRHKAKQMRFIDIIIGQVLTARTRLNVELPLTFMNSFHTSADTMKVLKHHRKFSQHDVPMEIIQHQEPKLVAATGEPVSYPANPELEWCPPGHGDLFSTIWESGLLDVLEERGFKYLFISNSDNLGARASRTLAQHFENTGAPFMAEVAIRTKADRKGGHIVRDKATGRLILREMSQVHPDDKEAAQDITKHPYFNTNSIWVRIDALKDKLAECDGVLPLPVIRNKKTVNPTDPDSEQVIQLETAMGAAIGLFNGSICVQVDRMRFLPVKTTNDLFIMRSDRFHLTDTYEMEDGNYIFPNVELDPRYYKNIHDFDERFPYAVPSLAAANSVSIQGDWTFGRDVMMFADAKLEDKGEPSYVPNGEYVGPQGIEPDDWV

SEQ ID NO: 5 - protein, 392 aa, Artificial Sequence
  Description of Artificial Sequence Synthetic polypeptide
MAQHLTTEALRKDFLAVFGQEADQTFFSPGRINLIGEHTDYNGGHVFPAAISLGTYGAARKRDDQVLRFYSANFEDKGIIEVPLADLKFEKEHNWTNYPKGVLHFLQEAGHVIDKGFDFYVYGNIPNGAGLSSSASLELLTGVVAEHLFDLKLERLDLVKIGKQTENNFIGVNSGIMDQFAIGMGADQRAIYLDTNTLEYDLVPLDLKDNVVVIMNTNKRRELADSKYNERRAECEKAVEELQVSLDIQTLGELDEWAVDQYSYLIKDENRLKRARHAVLENQRTLKAQVALQAGDLETFGRLMNASHVSLEHDYEVTGLELDTLVHTAWAQEGVLGARMTGAGFGGCAIALVQKDTVEAFKEAVGKHYEEVVGYAPSFYIAEVAGGTRVLD

SEQ ID NO: 6 - protein, 175 aa, Artificial Sequence
  Description of Artificial Sequence Synthetic polypeptide
MGLETVPAGKALPDDIYVVIEIPANSDPIKYEVDKESGALFVDRFMATAMFYPANYGYVNNTLSLDGDPVDVLVPTPYPLQPGSVIRCRPVGVLKMTDEAGSDAKVVAVPHSKLTKEYDHIKDVNDLPALLKAQIQHFFESYKALEAGKWVKVDGWEGVDAARQEILDSFERAKK

SEQ ID NO: 7 - DNA, 44 bp, Artificial Sequence
  Description of Artificial Sequence Synthetic primer
agctcatatggccgttattatttttgtgaatggtattcgtgccg

SEQ ID NO: 8 - DNA, 49 bp, Artificial Sequence
  Description of Artificial Sequence Synthetic primer
agctaagcttttacttctcttggccgaaaaactggttttcaatatctgc

SEQ ID NO: 9 - protein, 6 aa, Artificial Sequence
  Description of Artificial Sequence Synthetic 6xHis tag
HHHHHH

(19) United States
(12) Patent Application Publication
(10) Pub. No.: US 2022/0145343 A1
(43) Pub. Date: May 12, 2022

(54) MATERIALS AND METHODS FOR THE PREPARATION OF BACTERIAL CAPSULAR POLYSACCHARIDES

(71) Applicant: The Regents of the University of California, Oakland, CA (US)

(72) Inventors: Xi CHEN, Davis, CA (US); Riyao LI, Davis, CA (US); Hai YU, Woodland, CA (US)

(21) Appl. No.: 17/429,299

(22) PCT Filed: Feb. 7, 2020
(86) PCT No.: PCT/US2020/017321

§ 371 Date: Aug. 6, 2021

Related U.S. Application Data
(60) Provisional application No. 62/803,278, filed on Feb. 8, 2019.

(51) Int. Cl. C12P 19/04; A61K 39/095; A61K 39/102; A61K 39/09; A61K 39/108

(52) U.S. Cl. CPC C12P 19/04; A61K 39/095; A61K 39/0258; A61K 39/092; A61K 39/102

(57) ABSTRACT

Methods for preparing saccharide products such as bacterial capsular polysaccharides are provided. The methods include: forming a reaction mixture containing one or more bacterial capsular polysaccharide synthases, a sugar acceptor, and one or more sugar donors; and maintaining the reaction mixture under conditions sufficient to form the bacterial capsular saccharide product. Vaccine compositions containing bacterial capsular saccharide products prepared according to the methods are also described.

### CROSS REFERENCES TO RELATED APPLICATIONS

The present application claims priority to U.S. Provisional Pat. Appl. No. 62/803,278, filed on Feb. 8, 2019, which application is incorporated herein by reference in its entirety.

### STATEMENT AS TO RIGHTS TO INVENTIONS MADE UNDER FEDERALLY SPONSORED RESEARCH AND DEVELOPMENT

This invention was made with government support under Grant No. U01GM125288 awarded by the National Institutes of Health. The government has certain rights in the invention.

### BACKGROUND OF THE INVENTION

Neisseria meningitidis is a Gram-negative bacterium that causes diseases only for humans. Among 13 serogroups characterized so far based on the structures of their capsular polysaccharides (CPSs), six including serogroups A, B, C, W, X, and Y are causative agents of life-threatening meningococcal diseases. The CPSs for four (B, C, W, and Y) of these serogroups contain N-acetylneuraminic acid (Neu5Ac), the most common form of sialic acid (Sia) and a common terminal nine-carbon α-keto acid in humans. The CPSs of serogroups B and C are homopolymers of α2-8- and α2-9-linked Neu5Ac, respectively. In comparison, serogroups W and Y are heteropolymers of unique disaccharide repeating units -4Neu5Acα2-6Galα1- and -4Neu5Acα2-6Glcα1-, respectively, that have not been found in other organisms so far. The Neu5Ac in the CPSs of serogroups C, W, and Y can be modified by O-acetylation at C7 and C8 for serogroups C and at C7 and C9 for serogroups W and Y. The biosynthesis of these unusual polysaccharides is achieved by polymerases NmSiaD_{W} and NmSiaD_{y}. The genes encoding these proteins have been cloned, and the function of the expressed recombinant proteins has been confirmed by radiochemical assay and enzyme dissection. Claus, 2009; Claus 1997; Romanov, 2013; Romanov, 2014. However, the catalysis mechanism is not clearly understood.

N. meningitidis serogroup W has attracted an increasing attention after an outbreak after the Hajj pilgrimage in March 2000. Since 2009, increased cases of NmW infection with a high mortality rate (10% or higher) have been observed in the United Kingdom and the Netherlands. Despite the twentieth century's triumph in producing antibiotics for treating most bacterial infections, the continuous emergence of resistant strains of an increasing number of bacterial species has led to a focus on the development of vaccines. For Nm, CPSs have been valid targets for the development of glycoconjugate vaccines. Although protein-conjugated vaccines containing capsular polysaccharides of one or more serogroups of A, B, C, W, Y are available, there is a need for synthesizing structurally defined capsular polysaccharides for developing safer vaccines and as probes for basic research.

### BRIEF SUMMARY OF THE INVENTION

Provided herein are methods for preparing a bacterial capsular saccharide products. The methods include: forming a reaction mixture containing one or more bacterial capsular polysaccharide synthases, a sugar acceptor, and one or more sugar donors; and maintaining the reaction mixture under conditions sufficient to form the bacterial capsular saccharide product. The degree of polymerization of the bacterial capsular saccharide product ranges from 2 to about 200, and the polydispersity index M_{w}/Mₙ of the bacterial capsular saccharide product ranges from 1 to about 1.5.

Using the methods described herein, monodisperse heteropolymeric products and heterooligomeric products may be prepared in step-wise fashion or in one-step polymerization processes. The desired products may be conveniently prepared via one-step multienzyme reactions employing bacterial capsular polysaccharide synthases, such as N. meningitidis SiaD_{W}, in combination with further enzymes such as CMP-sialic acid synthetases, nucleotide sugar pyrophosphorylases, pyrophosphatases, and/or kinases.

Also provided herein are vaccine compositions containing the bacterial capsular saccharide products.

### BRIEF DESCRIPTION OF THE DRAWINGS

FIG. 1 shows an SDS-PAGE gel analysis of NmSiaD_{W} expression. Theoretical molecular weight of NmSiaD_{W} is 121.5 kDa. BI: before induction; AL: after induction; L: cell lysate; P: purified fraction.

FIG. 2 shows the chemical synthesis of sialylmonosaccharide S1 from N-acetylneuraminic acid (Neu5Ac, 1).

FIG. 3 shows the sequential one-pot multienzyme (OPME) chemoenzymatic synthesis of oligosaccharides G2-G10 from monosaccharide S1.

FIG. 4A shows galactosyltransferase activity and sialyltransferase activity across a range of pH values. Buffers used were: Citric acid, pH 3-4.5; MES, pH 5.0-6.5; Tris-HCl, pH 7.0-9.0; CAPS, pH 10.0-11.0.

FIG. 4B shows the effects of metals on galactosyltransferase activity and sialyltransferase activity.

FIG. 5A shows the thermostability profile of NmSiaD_{W}.

FIG. 5B shows the temperature profile of NmSiaD_{W}.

FIG. 6 shows initial velocity plots of Galα1-4Neu5AcαProNHCbz as acceptor with 2 mM or 10 mM of CMP-Neu5Ac as donor.

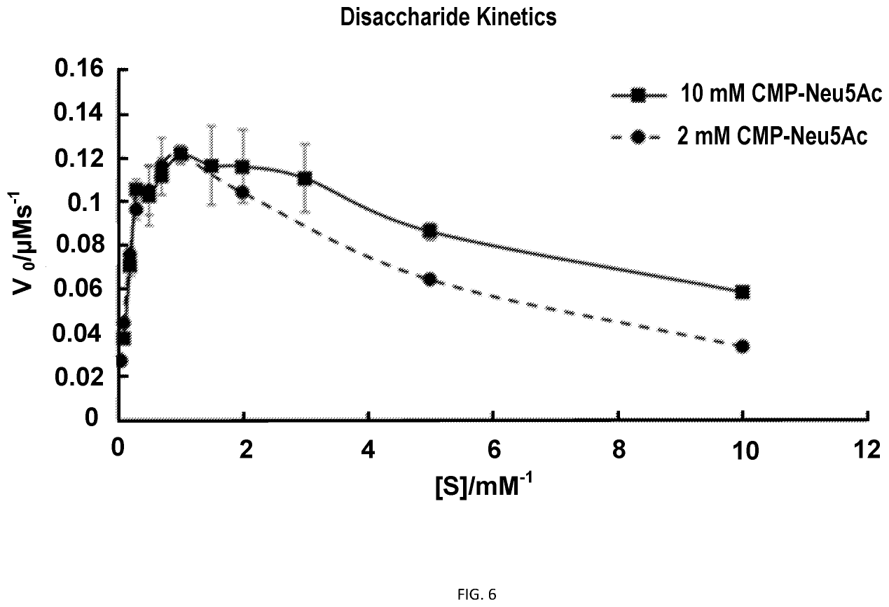

FIG. 7 shows the results of a polymerization study conducted with 10 oligosaccharide acceptors after 20 hour reaction.

FIG. 8A shows product profiles of 20-hour reactions using different ratios (1-50 equivalents) of donors versus acceptor (5 mM) where galactosyldisaccharide G2 was used as the acceptor.

FIG. 8B shows product profiles of 20-hour reactions using different ratios (1-50 equivalents) of donors versus acceptor (5 mM) where sialyltrisaccharide S3 was used as the acceptor.

### DETAILED DESCRIPTION OF THE INVENTION

Provided herein are methods for preparing bacterial capsular polysaccharides and other useful saccharide products. The methods include forming a reaction mixture containing an acceptor, a first sugar donor, a second sugar donor, and a bacterial capsular polysaccharide synthase; and maintaining the reaction mixture under conditions sufficient to form the saccharide product; wherein the first sugar donor is a sialic acid donor. Capsular polysaccharide synthases from pathogenic bacteria such as Neisseria meningitidis, Actinobacillus pleuropneumoniae, Haemophilus influenzae, Bibersteinia trehalosi, and Escherichia coli can be employed in the methods provided herein.

The chemoenzymatic methods of the present disclosure can avoid the contamination introduced by purifying capsular polysaccharides from pathogens. Furthermore, size-controlled oligosaccharides can be obtained using the methods described herein while avoiding the heterogeneity of the bacterial polysaccharide vaccines. Oligosaccharides can be synthesized using one-pot reactions with excellent yields, compared to previously reported chemical synthesis methods with multiple steps and lower yields. Both galactoside products and sialoside products can be obtained using the methods provided herein. Size-controlled oligosaccharides prepared according to the methods provided herein are advantageous for enzymology studies and improved vaccine development.

Some embodiments of the present disclosure provide highly active recombinant NmSiaD_{W} constructs that can be used in efficient one-pot multienzyme (OPME) sialylation and galactosylation systems for synthesizing size-controlled NmW CPS oligosaccharides and analogs. Recombinant NmSiaD_{W} can be cloned and expressed in E. coli with a high expression level (150 mg/L culture). In order to monitor the formation of oligosaccharides and facilitate the product purification process, a carboxybenzyl (Cbz) group can be introduced to the reducing end of Neu5Ac. Although NmW CPS sialosides have been synthesized by a total synthesis method, the present disclosure provides the first success in obtaining pure oligosaccharides in preparative-scale using chemoenzymatic methods. Structurally defined chromophore-tagged oligosaccharides allowed detailed characterization, kinetics studies, and substrate specificity studies of NmSiaD_{W}. The structurally-defined NmW CPS oligosaccharides synthesized can be employed as probes and carbohydrate standards, as well as for the development of improved bacterial carbohydrate-protein conjugate vaccines. The sequential OPME strategy can be extended for chemoenzymatic synthesis of other polysaccharides containing disaccharide repeating units.

### I. METHODS FOR PREPARATION OF BACTERIAL CAPSULAR OLIGOSACCHARIDES AND POLYSACCHARIDES

Provided herein are methods for preparing a bacterial capsular saccharide product.

The methods include:
forming a reaction mixture containing one or more bacterial capsular polysaccharide synthases, a sugar acceptor, and one or more sugar donors; and
maintaining the reaction mixture under conditions sufficient to form the bacterial capsular saccharide product;
wherein the degree of polymerization of the bacterial capsular saccharide product ranges from 2 to about 200, and wherein the polydispersity index M_{w}/Mₙ of the bacterial capsular saccharide product ranges from 1 to about 1.5.

A. Bacterial Capsular Saccharide Products

In some embodiments, the bacterial capsular saccharide product is a heteropolymer comprising disaccharide repeating units. Examples of disaccharide repeating units include, but are not limited to, -4GlcAβ1-4GlcNAcα1- (as expressed in capsular polysaccharides produce by microbes such as E. coli serotype K5; and P. multocida, Type D), -3GlcNAcβ1-4GlcAβ1- (as expressed in capsular polysaccharides produce by microbes such as P. multocida, Type A; and S. pyogenes), -3GalNAcβ1-4GlcAβ1- (as expressed in capsular polysaccharides produce by microbes such as P. multocida, Type F), -4Neu5Acα2-6Galα1- (as expressed in capsular polysaccharides produce by microbes such as N. meningitidis, serogroup W135), -4Neu5Acα2-6Glcα1- (as expressed in capsular polysaccharides produce by microbes such as N. meningitidis, serogroup Y), -3GlcAβ1-4Glcβ1- (as expressed in capsular polysaccharides produce by microbes such as S. pneumonia, Type 3), and -3GlcAβ1-4Glcβ1- (as expressed in capsular polysaccharides produce by microbes such as S. pneumonia, Type 37).

In some embodiments, the degree of polymerization (DP) of the bacterial capsular saccharide product ranges from 20 to about 200. The DP of a bacterial capsular polysaccharide product may range, for example, from about 20 to about 30, or from about 30 to about 40, or from about 40 to about 50, or from about 50 to about 60, or from about 60 to about 70, or from about 70 to about 80, or from about 80 to about 90, or from about 90 to about 100, or from about 100 to about 110, or from about 120 to about 130, or from about 130 to about 140, or from about 140 to about 150, or from about 150 to about 160, or from about 160 to about 170, or from about 170 to about 180, or from about 180 to about 190, or from about 190 to about 200. In some embodiments, the DP of a bacterial capsular polysaccharide may range from about from about 25 to about 115, or from about 30 to about 110, or from about 35 to about 105, or from about 40 to about 100, or from about 45 to about 95, or from about 50 to about 90, or from about 55 to about 85, or from about 60 to about 80, or from about 65 to about 75. In some embodiments, the DP of a bacterial capsular saccharide may from range about 5 to about 35, or from about 10 to about 30, or from about 15 to about 25.

The terms “about” and “around,” as used herein to modify a numerical value (e.g., degree of polymerization), indicate a close range surrounding that explicit value. If “X” were the value, “about X” or “around X” would indicate a value from 0.9X to 1.1X. “About X” thus includes, for example, a value from 0.95X to 1.05X, or from 0.98X to 1.02X, or from 0.99X to 1.01X. Any reference to “about X” or “around X” specifically indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.07X, 1.08X, 1.09X, and 1.10X. Accordingly, “about X” and “around X” are intended to teach and provide written description support for a claim limitation of, e.g., “0.98X.”

Advantageously, the methods of the present disclosure can be employed for the preparation of oligomeric and polymeric products having a narrow size distribution. Products having a degree of polymerization within the preceding ranges or subranges may be further characterized in terms of polydispersity index (PDI), calculated as M_{w}/Mₙ, wherein M_{w} is the weight average value of the population of polymers in the product and Mₙ is the number average value for the population of polymers in the product. Typically, the PDI for a bacterial capsular saccharide product will range from about 1 to about 1.5. The PDI may range, for example, from about 1.01 up to about 1.5, or from about 1.01 up to about 1.4, or from about 1.01 up to about 1.3, or from about 1.01 up to about 1.2, or from about 1 up to about 1.01, for a product have a PD value lying within any of the ranges or subranges set forth above. In some embodiments, the PDI of the bacterial capsular saccharide product is no greater than about 1.01, 1.02, 1.03, 1.04, 1.05, 1.06, 1.07, 1.09, 1.10, 1.11, 1.12, 1.13, 1.14, 1.15, 1.16, 1.17, 1.18, 1.19, or 1.20.

Weight average and number average molecular weights may be determined by any suitable method including, for example, by osmotic pressure, vapor pressure, light scattering, ultracentrifugation, or size exclusion chromatography. Using size exclusion chromatography with an appropriately calibrated column, number average molecular weight Mₙ may be determined according to Equation 1:

$${M}_{n} = \frac{W}{Σ {N}_{i}} = \frac{Σ ( {M}_{i} {N}_{i} )}{Σ {N}_{i}} = \frac{Σ ( {H}_{i} )}{Σ ( {H}_{i} / {M}_{i} )} , \qquad ( 1 )$$

and M_{w} may be determined according to Equation 2:

$${M}_{w} = \frac{Σ ( {W}_{i} {M}_{i} )}{W} = \frac{Σ ( {H}_{i} {M}_{i} )}{Σ {H}_{i}} , \qquad ( 2 )$$

wherein W is the total weight of polymers, Wᵢ is the weight of the i^{th} polymer, Mᵢ is the molecular weight of the i^{th} peak in a chromatogram, Nᵢ is the number of molecules with molecular weight Nᵢ, and Hᵢ is the height of the i^{th} peak in the chromatogram. Known polysaccharides and oligosaccharide, e.g., products characterized by NMR and HRMS as described below, may be employed in certain instances for calibration of instruments and analytical methodology.

In some embodiments, the degree of polymerization of the bacterial capsulate saccharide product is greater than 50. In some embodiments, the polydispersity index M_{w}/Mₙ of the bacterial capsular saccharide product ranges from 1.01 to about 1.15.

Methods according to the present disclosure generally include two or more glycosylation steps, which may be conducted with or without isolation of intermediates during elongation of the acceptor sugar toward the desire products. In one non-limiting grouping of embodiments, the methods employ alternating one-pot, multienzyme glycosylation reactions with product purification at the end of each reaction. This strategy can provide particularly precise control of product size distribution. For example, one-pot multienzyme galactose activation and transfer system (referred to as OPME1 in the Examples described below) can be used to add an α1-4-linked Gal to a sialoside acceptor (e.g., Neu5Ac-alpha-ProNHCbz) to form a product elongated by one additional sugar. The elongated product can be purified and used as a starting material for the one-pot multienzyme (OPME) sialic acid activation and transfer system (referred to as OPME2) to add an a2-6-linked sialic acid to form a subsequent product elongated by another additional sugar. This subsequent product can be purified and used for the next round of glycosylation. In this fashion, oligomeric and polymeric products can be prepared by alternating OPME1 and OPME2 reactions with product purification at the end of each reaction.

In another non-limiting grouping of embodiments, oligosaccharides (e.g., disaccharides to decasaccharides) may be employed as an initial sugar acceptor in the presence of two or more sugar donors (e.g., nucleotide sugars such as UDP-Gal and CMP-sialic acid) in a single polymerization step. The sugar donors may be provided externally or generated in situ by OPME reactions. This method can be used to provide products having a narrow range of molecular weights.

Accordingly, some embodiments of the present disclosure provide methods wherein forming the bacterial capsular saccharide product comprises glycosylating the sugar acceptor with monosaccharide residues of a first variety and monosaccharide residue of a second variety in alternating steps.

In some embodiments, forming the bacterial capsular saccharide product comprises glycosylating the sugar acceptor with alternating monosaccharide residues of a first variety and monosaccharide residues of a second variety in a single polymerization step.

Also provided are bacterial capsular saccharide products prepared according to the methods described herein.

B. Bacterial Capsular Polysaccharide Synthases

A number of bacterial capsular polysaccharide synthases may be used in the methods of the present disclosure. Suitable synthases include, but are not limited to, those described by Litschko et al. (mBio, 2018, 9(3): e00641-18). The synthases are generally characterized by glycosyltransferase activity, hexose-1-phosphate transferase activity, or a combination thereof.

Catalytic domains exhibiting glycosyltransferase activity typically adopt a “GT-A” fold or a “GT-B” fold. In the GT-A fold, two Rossmann-like domains are tightly associated, forming a central, continuous β-sheet. In the GT-B fold, two Rossmann-like domains are opposed to each other, forming a deep cleft that contains the catalytic center. Different mono-functional glycosyltransferases may also be employed in combination for the preparation of heteropolymeric or heterooligomeric products. Alternatively, synthases characterized by a two or more types of glycosyltransferase activity in the same polypeptide, e.g., N. meningitidis SiaD_{W}, may be employed in the preparation of heteropolymeric or heterooligomeric products. Synthases characterized by hexose-1-phosphate transferase can be used for the assembly of products containing sugar residues linked by phosphodiester moieties. Examples of such enzymes include, but are not limited to, N. meningitidis serogroup L CslB, and may be employed alone or with enzymes having glycosyltransferase activity.

In some embodiments, each bacterial capsular polysaccharide synthase is independently selected from N. meningitidis SiaD_{W} (NmSiaD_{W}; UniProt Accession No. 033390), N. meningitidis SiaD_{Y} (NmSiaD_{Y}; UniProt Accession No. B5WYL7), a P. multocida heparosan synthase (PmHS1 and PmHS2, GenBank Accession Nos. AAL84705 and AAQ55110), P. multocida hyaluronan synthase (PmHAS, GenBank Accession Nos. AAC38318), S. pyogenes hyaluronan synthase (SpHAS, GenBank Accession No. AAA17983), P. multocida chondroitin synthase (PmCS, GenBank Accession No. AAF97500), E. coli K5 KfiA and KfiC (GenBank Accession Nos. CAA54711 and CAA54709), S. pneumoniae Type 3 capsular polysaccharide synthase (SpCps3S, GenBank Accession No. CAA87404), and S. pneumoniae Type 37 capsular polysaccharide synthase (SpCps37Tts, GenBank Accession No. CAB51329).

In some embodiments, the bacterial capsular polysaccharide synthase may include one or more heterologous amino acid sequences located at the N-terminus and/or the C-terminus of the enzyme. The bacterial capsular polysaccharide synthase may contain a number of heterologous sequences that are useful for expressing, purifying, and/or using the enzyme. The bacterial capsular polysaccharide synthase can contain, for example, a poly-histidine tag (e.g., a His₆ tag, SEQ ID NO:9); a calmodulin-binding peptide (CBP) tag; a NorpA peptide tag; a Strep tag for recognition by/binding to streptavidin or a variant thereof, a FLAG peptide for recognition by/binding to anti-FLAG antibodies (e.g., M1, M2, M5); a glutathione S-transferase (GST); or a maltose binding protein (MBP) polypeptide.

In some embodiments, the reaction mixture comprises one bacterial capsular polysaccharide synthase, and the bacterial capsular polysaccharide synthase is NmSiaD_{W} having an amino acid sequence set forth in SEQ ID NO:1. In some embodiments, the bacterial capsular polysaccharide synthase is NmSiaD_{W} comprising a His6 tag, having an amino acid sequence set forth in SEQ ID NO:2.

In some embodiments, the bacterial capsular saccharide product comprises galactose-sialic acid disaccharide repeating units. In some embodiments, the galactose-sialic acid disaccharide repeating units are (-6Galα1-4Neu5Acα2). In some embodiments, NmSiaD_{W} is the bacterial capsular polysaccharide synthase employed for the preparation of products containing the galactose-sialic acid disaccharide repeating units.

C. Sugar Donors and Acceptors

Sugar donors used in the methods of the present disclosure typically contain a nucleotide bonded to a monosaccharide. Suitable nucleotides include, but are not limited to, adenine, guanine, cytosine, uracil and thymine nucleotides with one, two or three phosphate groups. The sugar can be any suitable sugar. Monosaccharides include, but are not limited to, glucose (Glc), glucosamine (2-amino-2-deoxy-glucose; GlcNH₂), N-acetylglucosamine (2-acetamido-2-deoxy-glucose; GlcNAc), galactose (Gal), galactosamine (2-amino-2-deoxy-galactose; GalNH₂), N-acetylgalactosamine (2-acetamido-2-deoxy-galactose; GalNAc), mannose (Man), mannosamine (2-amino-2-deoxy-mannose; ManNH₂), N-acetylmannosamine (2-acetamido-2-deoxy-mannose; ManNAc), glucuronic acid (GlcA), iduronic acid (IdoA), galacturonic acid (GalA), and sialic acids. Sialic acid is a general term for N- and O-substituted derivatives of neuraminic acid, and includes, but is not limited to, N-acetyl (Neu5Ac), N-glycolyl (Neu5Gc) derivatives, and 2-keto-3-deoxy-nonulosonic acid (Kdn), as well as O-acetyl, O-lactyl, O-methyl, O-sulfate and O-phosphate derivatives.

In some embodiments, the reaction mixture comprises a UDP-sugar, a CMP-sugar, or a combination thereof. In some embodiments, the reaction mixture comprises a galactose donor, a sialic acid donor, or a combination thereof. In some embodiments, the galactose donor is UDP-Gal. In some embodiments, the sialic acid donor is CMP-Neu5Ac.

Galactose donors such as UDP-Gal and sialic acid donors such as CMP-Neu5Ac may be used for forming bacterial capsular saccharide products by glycosylating the sugar acceptor with galactose residues and sialic acid residues in alternating steps as described above. Alternatively, galactose donors and sialic acid donors may be used for forming bacterial capsular saccharide products by glycosylating the sugar acceptor with alternating galactose residues and sialic acid residues in a single polymerization step. In some such embodiments, NmSiaD_{w} is used for the glycosylation steps.

Advantageously, the size and size distribution of desired products may be controlled by varying the sugar acceptor (e.g., varying the acceptor size and/or sugar composition) and the stoichiometry of the sugar donors and the sugar acceptors used in the glycosylation steps. Reaction stoichiometry may be adjusted based upon factors including, but not limited to, the desired degree of polymerization in the target product, the desired polydispersity index, or the kinetic parameters of the particular bacterial capsular polysaccharide synthase employed. As described in more detail below, for example, the catalytic efficiency of NmSiaD_{w} as an α1-4-galactosyltransferase has been found to depend, in part, on the size of the sugar acceptor whereas the catalytic efficiency of NmSiaD_{w} as an α2-6-sialyltransferase has been found to exhibit far less dependence on the size of the sugar acceptor. In addition, a narrower product size distribution can be achieved by using octasaccharide G8, nonasaccharide S9, or decasaccharide G10 as an acceptor substrate in polymerization reactions. Oligosaccharides, as opposed to monosaccharides, may therefore be preferred starting acceptor substrates for polymerization reactions depending on the nature of the target product. In this manner, the ratio [galactose donor:sialic acid donor:sugar acceptor] and the identity of the sugar acceptor in reactions employing capsular polysaccharide synthases such as NmSiaD_{q} may therefore be adjusted selected to provide products having a desired degree of polymerization and/or a desired polydispersity.

In some embodiments, the reaction mixture comprises the sugar donor(s) and the sugar acceptor in a ratio ranging from about 1:1 to about 250:1.

The ratio of the sugar donor(s) to the sugar acceptor may range, for example, from about 1:1 to about 25:1; or from about 25:1 to about 50:1; or from about 50:1 to about 75:1; or from about 75:1 to about 100:1; or from about 100:1 to about 125:1; or from about 125:1 to about 150:1; or from about 150:1 to about 175:1; or from about 175:1 to about 200:1; or from about 200:1 to about 225:1; or from about 225:1 to about 250:1.

The amount of the sugar donor in such ratios is intended to include the amount of a single sugar donor as well as the total amount of multiple sugar donors. As such, the ratios may be further differentiated as the ratio of a first sugar donor to a second sugar donor and a sugar acceptor, e.g., a ratio ranging from about 25:25:1 to about 25:50:1, or from about 30:45:1 to about 50:50:1. In some embodiments, the ratio can range from 25:25:1 to about 50:25:1, or from about 45:30:1 to about 50:50:1.

In some embodiments, the reaction mixture comprises UDP-Gal and CMP-Neu5Ac, and the ratio (UDP-Gal+CMP-Neu5Ac):(sugar acceptor) ranges from about 1:1 to about 250:1. In some embodiments, the ratio (UDP-Gal+CMP-Neu5Ac):(sugar acceptor) is about 100:1. In some embodiments, the ratio (UDP-Gal):(CMP-Neu5Ac):(sugar acceptor) is about 50:50:1.

A number of suitable acceptor sugars may be used in the methods provided herein. In some embodiments, the acceptor sugar contains a sialic acid (e.g., Neu5Ac) or a hexose (e.g., galactose) covalently bonded to a monosaccharide, an oligosaccharide, a polysaccharide, an amino acid, an oligopeptide, a polypeptide, a lipid, or another synthetic handle. In some embodiments, the sugar acceptor is a disaccharide, a trisaccharide, a tetrasaccharide, a pentasaccharide, a hexasaccharide, a heptasaccharide, an octasaccharide, a nonsaccharide, or a decasaccharide. In some embodiments, the sugar acceptor is an octasaccharide, a nonsaccharide, or a decasaccharide. The acceptor sugar may contain a purification handle, e.g., a hydrophobic moiety such as a perfluorinated alkyl group or a fatty acid moiety as described, for example, in WO 2014/201462. Products containing the purification handle may be separated from reaction mixtures via reverse phase chromatography, solid phase extraction, or like techniques. Purification handles may also include chromophores (e.g., aromatic substituents such as benzyloxycarbonyl) to aid in identification and purification of desired products. In some embodiments, the purification handle includes an (N-benzyloxycarbonyl)aminopropyl moiety. In some embodiments, the acceptor sugar has the structure:

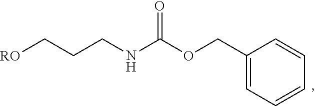

wherein R is a monosaccharide or an oligosaccharide. In some embodiments, R is an α- or β-linked Neu5Ac residue or an α- or β-linked galactose residue. In some embodiments R is an oligosaccharide moiety Galα1-4Neu5Acα2(-6Galα1-4Neu5Acα2)ₙ, wherein subscript n is 1, 2, 3, or 4. In some embodiments R is an oligosaccharide moiety Neu5Acα2(-6Galα1-4Neu5Acα2)ₘ, wherein subscript m is 1, 2, 3, 4, or 5. In some embodiments, the sugar acceptor comprises a sialic acid residue (e.g., an α- or β-linked Neu5Ac residue) at its non-reducing end. In some embodiments, the sugar acceptor comprises an α- or β-linked galactose residue at its non-reducing end.

Following purification, the benzxyloxycarbonyl moiety may be removed (e.g., by combination with an acid such a formic acid or trifluoroacetic acid) to provide an aminopropyl moiety —(CH₂)₃NH₂ at the reducing end of the bacterial capsular polysaccharide product. The aminopropyl moiety, in turn may serve as conjugation handle for covalent coupling to a carrier material, e.g., in a vaccine composition.

The methods generally include providing reaction mixtures that contain at least one bacterial capsular polysaccharide synthase, a sugar acceptor, and one or more sugar donors. The synthase can be, for example, isolated or otherwise purified prior to addition to the reaction mixture. As used herein, a “purified” enzyme refers to an enzyme which is provided as a purified protein composition wherein the enzyme constitutes at least about 50% of the total protein in the purified protein composition. For example, the enzyme can constitute about 50%, 60%, 70%, 75%, 80%, 85%, 90%, 95%, or 99% of the total protein in the purified protein composition. The amount of enzyme in a purified protein composition can be determined by any number of known methods including, for example, by polyacrylamide gel electrophoresis (e.g., SDS-PAGE) followed by detection with a staining reagent (e.g., Coomassie Brilliant Blue G-250, a silver nitrate stain, and/or a reagent containing a capsular polysaccharide antibody). The bacterial capsular polysaccharide synthases and other enzymes used in the methods can also be secreted by a cell present in the reaction mixture. Alternatively, a bacterial capsular polysaccharide synthase or other enzyme can catalyze the reaction within a cell expressing the enzyme.

Reaction mixtures can contain additional reagents for use in glycosylation techniques. For example, in certain embodiments, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, tetrahydrofuran, acetone, and acetic acid), salts (e.g., NaCl, KCl, CaCl₂, and salts of Mn²⁺ and Mg²⁺), detergents/surfactants (e.g., a non-ionic surfactant such as N,N-bis[3-(D-gluconamido)propyl]cholamide, polyoxyethylene (20) cetyl ether, dimethyldecylphosphine oxide, branched octylphenoxy poly(ethyleneoxy)ethanol, a polyoxyethylene-polyoxypropylene block copolymer, t-octylphenoxypolyethoxyethanol, polyoxyethylene (20) sorbitan monooleate, and the like; an anionic surfactant such as sodium cholate, N-lauroylsarcosine, sodium dodecyl sulfate, and the like; a cationic surfactant such as hexdecyltrimethyl ammonium bromide, trimethyl(tetradecyl) ammonium bromide, and the like; or a zwitterionic surfactant such as an amidosulfobetaine, 3-[(3-cholamidopropyl)dimethyl-ammonio]-1-propanesulfonate, and the like), chelators (e.g., ethylene glycol-bis(2-aminoethylether)-N,N,N′,N′-tetraacetic acid (EGTA), 2-({2-[Bis(carboxymethyl)amino]ethyl} (carboxymethyl)amino)acetic acid (EDTA), and 1,2-bis(o-aminophenoxy)ethane-N,N,N′,N′-tetraacetic acid (BAPTA)), reducing agents (e.g., dithiothreitol (DTT), β-mercaptoethanol (BME), and tris(2-carboxyethyl)phosphine (TCEP)), and labels (e.g., fluorophores, radiolabels, and spin labels). Buffers, cosolvents, salts, detergents/surfactants, chelators, reducing agents, and labels can be used at any suitable concentration, which can be readily determined by one of skill in the art. In general, buffers, cosolvents, salts, detergents/surfactants, chelators, reducing agents, and labels are included in reaction mixtures at concentrations ranging from about 1 μM to about 1 M. For example, a buffer, a cosolvent, a salt, a detergent/surfactant, a chelator, a reducing agent, or a label can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M. In some embodiments, the reaction mixture contains an acceptor sugar, one or more sugar donors, and a bacterial capsular polysaccharide synthase, as well as one or more components selected from a buffer, a cosolvent, a salt, a detergent/surfactant, a chelator, and a reducing agent. In some embodiments, the reaction mixture consists essentially of an acceptor sugar, one or more sugar donors, and a bacterial capsular polysaccharide synthase, as well as one or more components selected from a buffer, a cosolvent, a salt, a detergent/surfactant, a chelator, and a reducing agent.

Reactions are conducted under conditions sufficient to transfer the sugar of the sugar donor the acceptor sugar. The reactions can be conducted at any suitable temperature. In general, the reactions are conducted at a temperature of from about 4° C. to about 40° C. The reactions can be conducted, for example, at about 25° C. or about 37° C. The reactions can be conducted at any suitable pH. In general, the reactions are conducted at a pH of from about 4.5 to about 10. The reactions can be conducted, for example, at a pH of from about 5 to about 9, or from about 6 to about 9. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Other reaction conditions may be employed in the methods of the invention, depending on the identity of a particular bacterial capsular polysaccharide, sugar donor(s), or acceptor sugar.

D. One-Pot Multienzyme Reactions

Sugar donors such as sialic acid donors and galactose donors can be prepared prior to forming the bacterial capsular saccharide product, or the sugar donors can be prepared in situ immediately prior to formation of the bacterial capsular saccharide product. In some embodiments, the reaction mixture containing the bacterial capsular polysaccharide synthases further comprises one or more CMP-sialic acid synthetases, nucleotide sugar pyrophosphorylases, pyrophosphatases, kinases, or combinations thereof. The enzymes may be employed in one-pot reactions for convenient preparation of the desired bacterial capsular saccharide products.

In some embodiments, the methods include enzymatic preparation of sialic acid donors such as CMP-Neu5Ac. In some embodiments, the methods include forming a reaction mixture including a CMP-sialic acid synthetase, cytidine triphosphate, and N-acetylneuraminic acid (Neu5Ac) or a Neu5Ac analog, under conditions suitable to form CMP-Neu5Ac or a CMP-Neu5Ac analog. Any suitable CMP-sialic acid synthetase (i.e., N-acetylneuraminate cytidylyltransferase, EC 2.7.7.43) can be used in the methods of the invention. For example, CMP-sialic acid synthetases from E. coli, C. thermocellum, S. agalactiae, P. multocida, H. ducreyi, or N. meningitidis can be used. In some embodiments, the CMP-sialic acid synthetase is NmCSS, having an amino acid sequence set forth in SEQ ID NO:3.

In some embodiments, the sialic acid moiety of the sialic acid donor is prepared separately prior to use in the methods. Alternatively, the sialic acid moiety can be prepared in situ immediately prior to use in the methods. In some embodiments, the methods include forming a reaction mixture including a sialic acid aldolase, pyruvic acid or derivatives thereof, and N-acetylmannosamine or derivatives thereof, under conditions suitable to form Neu5Ac or a Neu5Ac analog. Any suitable sialic acid aldolase (i.e., N-acetylneuraminate pyruvate lyase, EC 4.1.3.3) can be used. For example, sialic acid aldolases from E. coli, L. plantarum, P. multocida, or N. meningitidis can be used.

In some embodiments, the methods include enzymatic preparation of sialic acid donors such as UDP-Gal. In some embodiments, the methods include forming a reaction mixture including a nucleotide sugar pyrophosphorylase, uridine triphosphate, and optionally a kinase, dehydrogenase, and/or a pyrophosphatase, and maintaining the mixture under conditions suitable to form UDP-Gal. The nucleotide sugar pyrophosphorylase may be, for example, a glucosamine uridyltransferase (GlmU), a Glc-1-P uridylyltransferase (GalU), or a promiscuous UDP-sugar pyrophosphorylase (USP). In some embodiments, GlmU from P. multocida (PmGlmU) may be employed. Suitable GalUs can be obtained, for example, from yeasts such as Saccharomycesfragilis, pigeon livers, mammalian livers such as bovine liver, Gram-positive bacteria such as Bifidobacterium bifidum, and Gram-negative bacteria such as Echerichia coli (EcGalU) (Chen X, Fang J W, Zhang J B, Liu Z Y, Shao J, Kowal P, Andreana P, and Wang P G. J. Am. chem. Soc. 2001, 123, 2081-2082). In some embodiments, the nucleotide-sugar pyrophosporylase is a USP. USPs include, but are not limited to, those obtained from Pisum sativum L. (PsUSP) and Arabidopsis thaliana (AtUSP), as well as enzymes obtained from protozoan parasites (such as Leishmania major and Trypanosoma cruzi) and hyperthermophilic archaea (such as Pyrococcusfuriosus DSM 3638). USPs also include human UDP-GalNAc pyrophosphorylase AGX1, E. coli EcGlmU, and Bifidobacterium longum BLUSP. In some embodiments, the nucleotide sugar pyrophosphorylase is BLUSP, having an amino acid sequence set forth in SEQ ID NO:4.

The reaction mixture may also contains a kinase or a dehydrogenase. The kinase may be, for example, an N-acetylhexosamine 1-kinase (NahK), a galactokinase (GalK), or a glucuronokinase (GlcAK). In some embodiments, the kinase is an NahK. The NahK can be, for example, Bifidobacterium infantis NahK_ATCC15697 or Bifidobacterium longum NahK_ATCC55813. NahK_ATCC15697 and NahK_ATCC55813 were cloned and characterized by the inventors. In some embodiments, the kinase is a GalK. The GalK can be, for example, Escherichia coli EcGalK (Chen X, Fang J W, Zhang J B, Liu Z Y, Shao J, Kowal P, Andreana P, and Wang P G. J. Am. chem. Soc. 2001, 123, 2081-2082) and Streptococcus pneumoniae TIGR4 SpGalK (Chen M, Chen L L, Zou Y, Xue M, Liang M, Jing L, Guan W Y, Shen J, Wang W, Wang L, Liu J, and Wang P G. Carbohydr. Res. 2011, 346, 2421-2425). In some embodiments, the kinase is SpGalK, having an amino acid sequence set forth in SEQ ID NO:5.

The reaction mixture formed in the methods of the invention can further include an inorganic pyrophosphatase (PpA). PpAs can catalyze the degradation of the pyrophosphate (PPi) that is formed during the conversion of a sugar-1-phosphate to a UDP-sugar. PPi degradation in this manner can drive the reaction towards the formation of the UDP-sugar products. The pyrophosphatase can be, but is not limited to, Pasteurella multocida PmPpA (Lau K, Thon V, Yu H, Ding L, Chen Y, Muthana M M, Wong D, Huang R, and Chen X. Chem. Commun. 2010, 46, 6066-6068). In some embodiments, the inorganic pyrophosphatase is PmPpA, having an amino acid sequence set forth in SEQ ID NO:6.

Enzymes employed in the methods of the present disclosure, including bacterial capsular polysaccharide synthases, CMP-sialic acid synthetases, nucleotide sugar pyrophosphorylases, pyrophosphatases, and/or kinases, may include amino acid sequences characterized by varying levels of sequence identity to any of the exemplary enzyme sequences set forth above. The amino acid sequence of a particular enzyme may have, for example, at least about 70%, e.g., at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of the amino acid sequences set forth in SEQ ID NOS:1-6.

“Identical” and “identity,” in the context of two or more polypeptide sequences, refer to two or more sequences or subsequences that are the same. Sequences are “substantially identical” to each other if they have a specified percentage of nucleotides or amino acid residues that are the same (e.g., at least 75%, at least 80%, at least 85%, at least 90%, at least 95%, or at least 99% identical over a specified region), when compared and aligned for maximum correspondence over a comparison window, or designated region as measured using one of the following sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). Additional examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available, for example, at the National Center for Biotechnology Information website. The BLAST algorithm parameters W, T, and X determine the sensitivity and speed of the alignment. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

In some embodiments, the CMP-sialic acid synthetase, the nucleotide sugar pyrophosphorylase, the pyrophosphatase, and/or the kinase may be purified as described above. Other components (e.g., buffers, cosolvents, salts, detergents/surfactants, chelators, and/or reducing agents, as described above) can be included in the reaction mixture for forming the CMP-Neu5Ac and/or UDP-Gal. In some embodiments, the step of forming the galactose donor, the sialic acid donor, the sialic acid moiety of the sialic acid donor, and/or the step of forming the bacterial capsular saccharide product are performed in one pot. In some embodiments, the pH of the one-pot multienzyme reaction mixture ranges from about 6 to about 9. In some embodiments, the method is conducted in vitro.

### II. VACCINE COMPOSITIONS

Also provided herein are vaccine compositions. The compositions contain one or more bacterial capsular saccharide products, include products prepared according to the method described herein, coupled to a carrier material. A vaccine composition according to the present disclosure can be used, for example, as an N. meningitidis serogroup W vaccine. Examples of carrier materials include, but are not limited to, carrier proteins such as a genetically modified cross-reacting material (CRM197) of diphtheria toxin, tetanus toxoid (TT), meningococcal outer membrane protein complex (OMPC), diphtheria toxoid (DD), and H. influenzae protein D (HiD). See, e.g., Pichichero (Human Vaccines & Immunotherapeutics 2013, 9(12): 2505-2523) and Berti et al. (Chem. Soc. Rev., 2018, 47, 9015-9025), which are incorporated herein by reference in their entirety.

Bacterial capsular saccharide products can be covalently bonded to proteins and other carrier materials using various chemistries for protein modification. A wide variety of such reagents are known in the art. Examples of such reagents include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Crosslinking reagents can react to form covalent bonds with functional groups in the bacterial capsular saccharide product (e.g., an aminopropyl group as described above) and in a protein or other carrier material (e.g., a primary amine, a thiol, a carboxylate, a hydroxyl group, or the like). Crosslinkers useful for attaching bacterial capsular saccharide products to proteins and other carrier materials include homobifunctional crosslinkers, which react with the same functional group in the bacterial capsular saccharide product and the carrier, as well as heterobifunctional crosslinkers, which react with functional groups in the bacterial capsular saccharide product and the carrier that differ from each other.

Examples of homobifunctional crosslinkers include, but are not limited to, amine-reactive homobifunctional crosslinkers (e.g., dimethyl adipimidate, dimethyl suberimidate, dimethyl pimilimidate, disuccinimidyl glutarate, disuccinimidyl suberate, bis(sulfosuccinimidyl) suberate, bis(diazo-benzidine), ethylene glycobis(succinimidyl-succinate), disuccinimidyl tartrate, disulfosuccinimidyl tartrate, glutaraldehyde, dithiobis(succinimidyl propionate), dithiobis-(sulfosuccinimidyl propionate), dimethyl 3,3′-dithiobispropionimidate, bis 2-(succinimidyl-oxycarbonyloxy)ethyl-sulfone, and the like) as well as thiol-reactive homobifunctional crosslinkers (e.g., bismaleidohexane, 1,4-bis-[3-(2-pyridyldithio)propionamido]butane, and the like). Examples of heterobifunctional crosslinkers include, but are not limited to, amine- and thiol-reactive crosslinkers (e.g., succinimidyl 4-(N-maleimidomethyl)-cyclohexane-1-carboxylate, m-maleimidobenzoyl-N-hydroxysuccinimide ester, succinimidyl-4-(p-maleimidophenyl)butyrate, N-(γ-maleimidobutyryloxy)succinimide ester, N-succinimidyl(4-iodoacetyl) aminobenzoate, 4-succinimidyl oxycarbonyl-α-(2-pyridyldithio)-toluene, sulfosuccinimidyl-6-α-methyl-α-(2-pyridyldithio)-toluamido-hexanoate, N-succinimidyl-3-(2-pyridyldithio) propionate, N-hydroxysuccinimidyl 2,3-dibromopropionate, and the like). Further reagents include but are not limited to those described in Hermanson, Bioconjugate Techniques 2nd Edition, Academic Press, 2008.

Vaccine compositions, or compositions thereof, can be administered to a subject by any of the routes normally used for administration of vaccines. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local. Appropriate pharmaceutically acceptable carriers can be selected based on facts including, but not limited to, the particular composition being administered, as well as by the particular method used to administer the composition.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

In some embodiments, the vaccine composition is sufficiently immunogenic as a vaccine for effective immunization without administration of an adjuvant. In some embodiments, immunogenicity of a composition is enhanced by including an adjuvant. Any adjuvant may be used in conjunction with the vaccine composition. A large number of adjuvants are known; see, e.g., Allison, 1998, Dev. Biol. Stand., 92:3-11, Unkeless et al., 1998, Annu. Rev. Immunol., 6:251-281, and Phillips et al., 1992, Vaccine, 10:151-158. Exemplary adjuvants include, but are not limited to, cytokines, gel-type adjuvants (e.g., aluminum hydroxide, aluminum phosphate, calcium phosphate, etc.), microbial adjuvants (e.g., immunomodulatory DNA sequences that include CpG motifs; endotoxins such as monophosphoryl lipid A; exotoxins such as cholera toxin, E. coli heat labile toxin, and pertussis toxin; muramyl dipeptide, etc.), oil-emulsion and emulsifier-based adjuvants (e.g., Freund's Adjuvant, MF59 [Novartis], SAF, etc.), particulate adjuvants (e.g., liposomes, biodegradable microspheres, saponins, etc.), synthetic adjuvants (e.g., nonionic block copolymers, muramyl peptide analogues, polyphosphazene, synthetic polynucleotides, etc.) and/or combinations thereof.

### III. EXAMPLES

Chemicals were purchased and used as received without further purification. NMR spectra were recorded in the NMR facility of the University of California using a VNMRS-600 NMR spectrometer (600 MHz for ¹H, 150 MHz for ¹³C) or a 800 MHz Bruker Avance III spectrometer. Chemical shifts are reported in parts per million (ppm) on the 6 scale. High resolution (HR) electrospray ionization (ESI) mass spectra were obtained using a Thermo Electron LTQ-Orbitrap Hybrid MS at the Mass Spectrometry Facility in the University of California, Davis. UPLC detections were assayed using Agilent 1290 Infinity LC with an EclipsePlus C18 (Rapid Resolution HD, 1.8 μm, 2.1×50 mm, 959757-902) or an AdvanceBio Glycan Map column (1.8 μm, 2.1×150 mm, 859700-913) column from Agilent Technologies. Reverse phase chromatography was performed with C18 column (ODS-SM, 50 mm, 120 Å, 3.0×20 cm) from Yamazen Corporation on a CombiFlash Rf 200i system. Galactose was from Fisher Scientific. N-Acetylneuraminic acid (Neu5Ac) was from Inalco (Italy). Adenosine 5′-triphosphate (ATP), cytosine 5′-triphosphate (CTP) and uridine 5′-triphosphate (UTP) were purchased from Hangzhou Meiya Pharmaceutical Co. Ltd. UTP was also purchased from Chemfun Medical Technology Co. ATP was also purchased from Beta Pharm Inc.

Recombinant enzymes Neisseria meningitidis CMP-sialic acid synthetase (NmCSS), Pasteurella multocida inorganic pyrophosphatase (PmPpA), Streptococcus pneumoniae TIGR4 galactokinase (SpGalK), Bifidobacterium longum UDP-sugar pyrophosphorylase (BLUSP) were expressed and purified as described previously. See: Yu, H. et al. Bioorg. Med. Chem. 2004, 12, 6427-6435; Lau, K. et al. Chem. Commun. 2010, 46, 6066-6068; Chen, M. et al. Carbohydr. Res. 2011, 346, 2421-2425; and Muthana, M. et al. Chem. Commun. 2012, 48, 2728-2730. Neu5AcαProNHCbz was prepared as described previously.

### Example 1. Cloning and Expression of NmSiaD_{W}

A. Overexpression and Purification

The NmSiaD_{W} gene (GenBank accession number Y13970) with sequence optimized for expression in E. coli was custom synthesized by GeneArt and cloned in pMA-RQ (ampR) vector. To subclone NmSiaD_{W} as an C-His₆-tagged fusion protein in pET22b(+) vector, two primers were designed for polymerase chain reaction (PCR) and the sequences were: forward primer:

5′-AGCTCATATGGCCGTTATTATTTTTGTG
AATGGTATTCGTGCCG-3′
(SEQ ID NO: 7, NdeI restriction
site is italicized);
and
reverse primer:
5′-AGCTAAGCTTTTACTTCTCTTGGCCGAA
AAACTGGTTTTCAATATCTGC-3′
(SEQ ID NO: 8, HindIII restriction
site is italicized).

PCR was performed in a 50-μL reaction mixture containing plasmid DNA (50 ng), forward and reverse primers (0.5 μM each), 5×reaction buffer (10 μL), dNTP mixture (0.2 mM), and 1 U of Phusion High-Fidelity DNA Polymerase (New England Biolabs). The reaction mixture was subjected to 30 cycles of amplification with an annealing temperature of 72° C. The resulting PCR product was purified and digested with NdeI and HindIII restriction enzymes. The purified and digested PCR product was ligated with a predigested pET22b(+) vector and transformed into E. coli DH5a cells. Selected clones were grown for minipreps and the purified plasmids were analyzed by DNA sequencing performed by Genewiz.

A plasmid with confirmed sequence was transformed into Escherichia coli BL21 (DE3). To express the recombinant NmSiaD_{W}, bacteria were cultivated in 1 L of LB rich medium in the presence of 100 μg/mL ampicillin. The expression was achieved by induction with 0.1 mM of isopropyl β-D-1-thiogalactopyranoside (IPTG) when OD_{600 nm} of the culture reached 0.6 followed by incubation at 16° C. for 72 h. Cells were harvested (6000×g, 15 min, 16° C.), re-suspended in lysis buffer (50 mM Tris-HCl, pH 8.0, 300 mM NaCl, 0.1% Triton X-100), and the mixture was subjected to sonication (amplitude 60%, 3 s on and 15 s off, 6 min). The supernatant was obtained by centrifugation (4300×g, 30 min, 4° C.), loaded onto a Ni²⁺-NTA affinity column at 4° C. that was pre-equilibrated with 6 column volumes of binding buffer containing Tris-HCl buffer (50 mM, pH 8.0), NaCl (300 mM), and imidazole (5 mM). It was washed with 10 column volumes of binding buffer and 10 column volumes of 10% and 20% elute buffer, respectively, and eluted with elute buffer containing Tris-HCl (50 mM, pH 8.0), NaCl (300 mM), imidazole (150 mM). The purified protein fractions were combined and concentrated. The resulting sample was dialyzed using dialysis buffer (Tris-HCl, 20 mM, pH 8.0 containing 10% glycerol) and stored at 4° C.

B. Sodium Dodecylsulfate-Polyacrylamide Gel Electrophoresis (SDS-PAGE) Analysis.

Gels were prepared with 12% acrylamide in the presence of 0.1% SDS. Cells and protein samples were incubated in the loading buffer (50 mM Tris-HCl, pH 6.8, 0.1% bromophenol blue, 10% glycerol, 100 mM DTT) for 10 min at 95° C. Denatured samples were loaded to the gel and the gel was developed at 150 V for 1 h. The gel was then stained with coomassie blue dye (1 g/L) in a solution of acetic acid:methanol:water (=1:4:5 by volume) and de-stained using the same solution without the dye.

C. Results

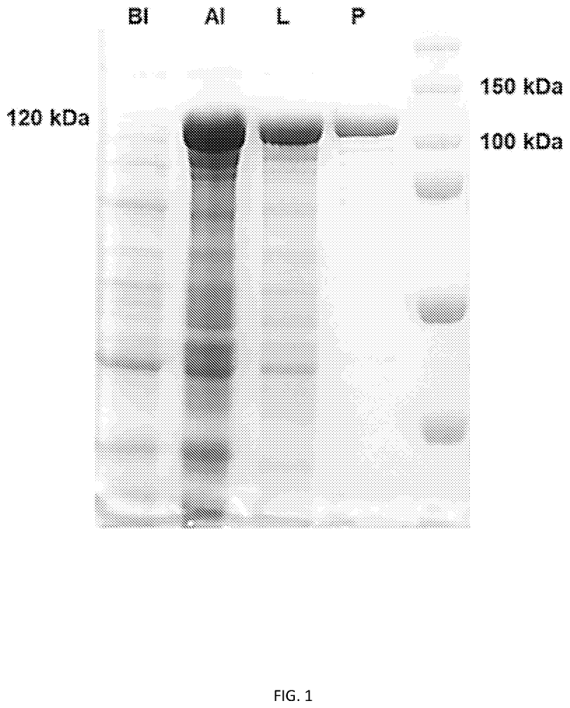

NmSiaD_{W} has been cloned and expressed in Escherichia coli previously. In our attempts, initial cloning into pET15b vectors led to a low expression of soluble and active enzymes in Escherichia coli BL21 (DE3) cells. In order to improve the protein expression, NmSiaD_{W} was recombined to pET22b (+) vectors and expressed as a C-terminal His-tagged protein. Soluble and active enzymes could be obtained by inducing Escherichia coli BL21 (DE3) cells with 0.1 mM of isopropyl β-D-1-thiogalactopyranoside (IPTG) followed by incubation at 16° C. for 72 hours. Purification was achieved by one-step nickel-nitrilotriacetic acid (Ni²⁺-NTA) affinity chromatography. About 150 mg of NmSiaD_{W} could be obtained from 1 liter LB Broth cell culture. Sodium dodecylsulfate-polyacrylamide gel electrophoresis (SDS-PAGE) analysis indicated that the apparent molecular weights of purified NmSiaD_{W}(FIG. 1) were about 120 kDa, which were consistent with their calculated molecular weights of 121.5 kDa. In our practice, NmSiaD_{W} barely dissolved in the lysis buffer without detergent, but solubility greatly increased with 0.1% Triton X-100 in the lysis buffer, indicating the association of the enzyme with membranes.

### Example 2. Preparative-Scale One-Pot Multienzyme Synthesis

A. General Procedure for Preparative Synthesis of Sialosides Using NmSiaD_{W}

Reactions were performed in the presence of 5 mM acceptor (monosaccharide to decasaccharide), 50 mM UDP-Gal, 50 mM CMP-Neu5Ac, 100 mM Tris-HCl, pH 8.0, 10 mM MgCl₂ and 50 μg NmSiaD_{W} with a total volume of 50 μL. Reactions were performed in duplicate at 30° C. After 1 h, 20 μL reaction mixture was quenched by addition of 20 μL pre-chilled ethanol and incubated at −20° C. for 30 min before detection. After 20 h, another 20 μL reaction mixture was quenched by addition of 20 μL pre-chilled ethanol and incubated at −20° C. for 30 min before detection. Products were detected using UPLC with AdvanceBio Glycan Map column, Agilent. Sample was eluted with a gradient from 90% to 50% acetonitrile in 25 minutes.

B. General Procedure for One-Pot Two-Enzyme Preparative Synthesis of Sialoside

Galactoside acceptor (1.0 equiv, 10 mM), CTP (1.3 equiv) and Neu5Ac (1.3 equiv) were dissolved in water containing 100 mM Tris-HCl, pH 8.5 and 20 mM MgCl₂. After addition of appropriate amounts NmCSS (0.5-4 mg), NmSiaD_{W} (0.5-4 mg), water was added to bring the final volume of reaction mixture to 15-64 mL. The reaction was carried out by incubating the solution in an incubator for 20 h at 30° C. with agitation at 100 rpm. Product formation was monitored by UPLC (EclipsePlusC18 column or AdvanceBio Glycan Map column, Agilent). The reaction was quenched by adding the same volume of pre-chilled methanol and incubation at −20° C. for 30 min. The supernatant was concentrated and purified by a C18 column. Water with 0.1% TFA (v/v) and acetonitrile were used as solvents with a gradient. The fraction that containing the product were collected, neutralized, concentrated and further purified by a C18 column. Water and acetonitrile were used as solvents with a gradient. Products were purified as sodium salts.

C. General Procedures for One-Pot Four-Enzyme Preparative Synthesis of Galactoside

Sialoside acceptor (1.0 equiv, 10 mM), UTP (1.3 equiv), ATP (1.3 equiv) and galactose (1.3 equiv) were dissolved in water containing 100 mM Tris-HCl, pH 8.5 and 20 mM MgCl₂. After addition of appropriate amounts of SpGalK (1.0-8.5 mg), BLUSP (1.0-8.5 mg), PmPpA (1.0-8.0 mg), NmSiaD_{W} (0.5-8.5 mg), water was added to bring the final volume of reaction mixture to 10-80 mL. The reaction was carried out by incubating the solution in an incubator for 20 h at 30° C. with agitation at 100 rpm. Product formation was monitored by UPLC (EclipsePlusC18 column or AdvanceBio Glycan Map column, Agilent). The reaction was quenched by adding the same volume of pre-chilled methanol and incubation at −20° C. for 30 min. The supernatant was concentrated and purified by a C18 column. Water with 0.1% TFA (v/v) and acetonitrile were used as solvents with a gradient. The fraction that containing the product were collected, neutralized, concentrated and further purified by a C18 column. Water and acetonitrile were used as solvents with a gradient. Products were purified as sodium salts.

D. pH Profile

Assays were carried out in duplicate at 30° C. for 20 min in a total volume of 10 μL in a buffer (200 mM) with a pH value in the range of 3.0-11.0 containing a donor substrate (1.2 mM) (UDP-Gal for GalT and CMP-Neu5Ac for SiaT assays), an acceptor substrate (1 mM) (S1 for GalT and G2 for SiaT assays), MgCl₂ (10 mM), and NmSiaD_{W} (19.8 μg for GalT and 0.17 μg for SiaT assays). Reactions were quenched by adding 10 μL of pre-chilled ethanol followed by incubation at −20° C. for 30 min. The precipitates were removed by centrifugation (11000×g, 5 min, 4° C.). Reaction mixtures were assayed using an Agilent 1290 Infinity II LC System with a PDA detector (monitored at 215 nm) and an Eclipse Plus C18 column (Rapid Resolution HD, 1.8 μm, 2.1×50 mm, 959757-902) at 30° C. An elution solvent of 11% acetonitrile and 89% H₂O containing 0.1% TFA was used for S1 and 10% acetonitrile and 90% H₂O containing 0.1% TFA was used for G2. Buffers used were: Citric acid, pH 3.0-4.5; MES, pH 5.0-6.5; Tris-HCl, pH 7.0-9.0; CAPS, pH 10.0-11.0.

E. Metal Effects Screening

Assays were carried out in duplicate at 30° C. for 20 min in a total volume of 10 μL in a buffer (MES, 100 mM, pH 6.5 for GalT and Tris-HCl, 100 mM, pH 8.0 for SiaT assays) containing a donor substrate (1.2 mM) (UDP-Gal for GalT and CMP-Neu5Ac for SiaT assays), an acceptor substrate (1 mM) (S1 for GalT and G2 for SiaT assays), NmSiaD_{W} (19.8 μg for GalT and 0.17 μg for SiaT assays), and the presence of EDTA, DTT, Mg²⁺, Ca²⁺, Li⁺, Na⁺, Co²⁺, Cu²⁺, Mn²⁺, or Ni²⁺ (10 mM). Reactions were quenched by adding 10 μL of pre-chilled ethanol followed by incubation at −20° C. for 30 min. Reaction mixtures were assayed as described above for pH profile studies.

F. Temperature Profile

Assays were carried out in duplicate at different temperatures for 20 min in a total volume of 10 μL in a buffer (MES, 100 mM, pH 6.5 for GalT and Tris-HCl, 100 mM, pH 8.0 for SiaT assays) containing a donor substrate (1.2 mM) (UDP-Gal for GalT and CMP-Neu5Ac for SiaT assays), an acceptor substrate (1 mM) (S1 for GalT and G2 for SiaT assays), MgCl₂ (10 mM), and NmSiaD_{W} (19.8 μg for GalT and 0.17 μg for SiaT assays). Reactions were quenched by adding 10 μL of pre-chilled ethanol to the reaction mixture followed by incubation at −20° C. for 30 min. Products were assayed as described above for pH profile studies.

G. Thermostability

Enzyme was pre-heated at a given temperature for 30 min, then put on ice for 10 min. Reactions were performed at 30° C. and activity assays were then carried out as described above for the temperature profile assays.

H. Results

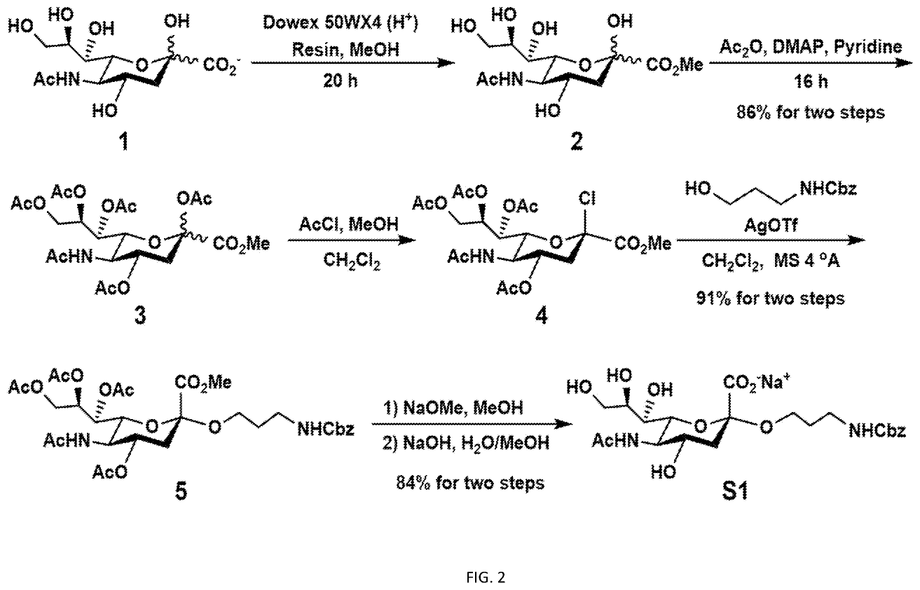

To facilitate the enzyme characterization and product purification, a chromophore-tagged substrate was designed. 2-O—(N-Benzyloxycarbonyl)aminopropyl α-N-acetylneuraminide (Neu5AcαProNHCbz, S1 for sialyl monosaccharide) was chemically synthesized from Neu5Ac (FIG. 2) in a process similar to that reported previously. See, Sardzik 2011. Briefly, methylation of the carboxyl group in the commercially available Neu5Ac (1) followed by peracetylation produced per-O-acetylated Neu5Ac methyl ester (3) in 86% yield. Treatment of 3 with acetyl chloride in dichloromethane and anhydrous methanol formed per-O-acetylated Neu5Ac chloride (4), which was reacted with benzyl N-(3-hydroxypropyl) carbamate in the presence of AgOTf to produce protected Neu5Ac glycoside (5) in an excellent 91% yield. De-O-acetylation using NaOMe in MeOH and hydrolysis of methyl ester using sodium hydroxide produced the desired product Neu5AcαProNHCbz (S1, 3.79 g) in 84% yield.

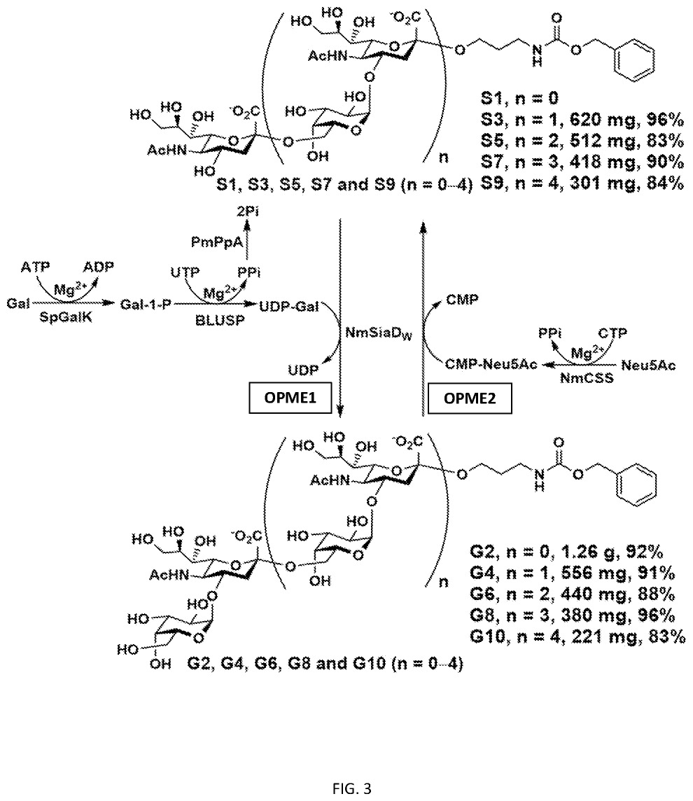

To elongate Neu5AcαProNHCbz (S1) for the synthesis of galactosyl disaccharide Galα1-4Neu5AcαProNHCbz (G2), an OPME α1-4-galactosylation system containing Streptococcus pneumoniae TIGR4 galactokinase (SpGalK) [Chen, M. 2011], Bifidobacterium longum UDP-sugar pyrophosphorylase (BLUSP) [Muthana, 2012], Pasteurella multocida inorganic pyrophosphatase (PmPpA) [Lau, 2010], and NmSiaD_{W} was applied. In this system, SpGalK was used to phosphorylate the anomeric position of galactose. BLUSP catalyzed the formation of the activated sugar nucleotide donor UDP-Gal from galactose-1-phosphate and uridine 5′-triphosphate (UTP). PmPpA catalyzed the hydrolysis of inorganic pyrophosphate to drive the reaction forward. Finally, NmSiaD_{W} catalyzed the transfer of galactose to the sialoside (FIG. 3).

To sialylate the obtained disaccharide G2 to form sialyl trisaccharide Neu5Acα2-6Galα1-4Neu5AcαProNHCbz (S3), an OPME α2-6-sialylation system containing Neisseria meningitidis CMP-sialic acid synthetase (NmCSS) [Yu 2004] and NmSiaD_{W} was used. In this system, NmCSS catalyzed the formation of the activated sugar nucleotide donor CMP-Neu5Ac from CTP and Neu5Ac for NmSiaD_{W}-catalyzed sialylation reaction (Scheme 2).

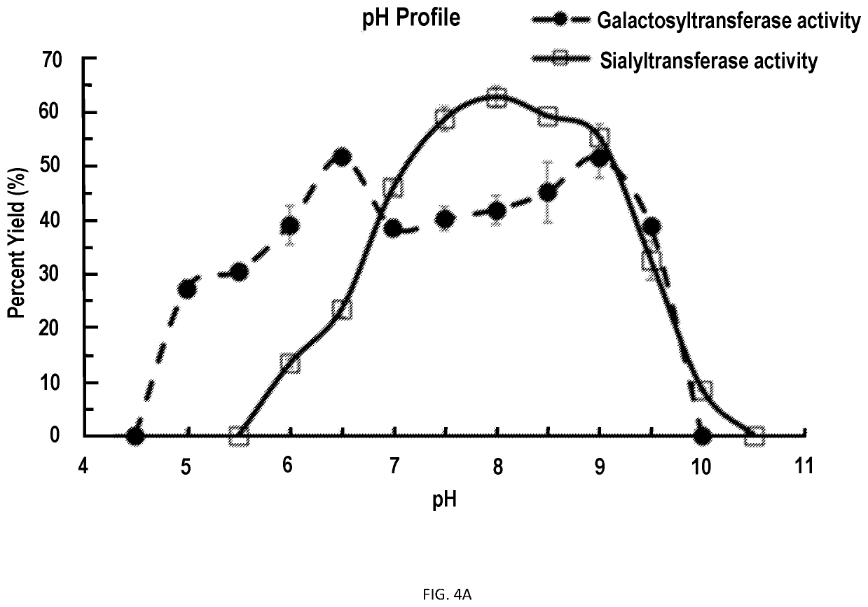

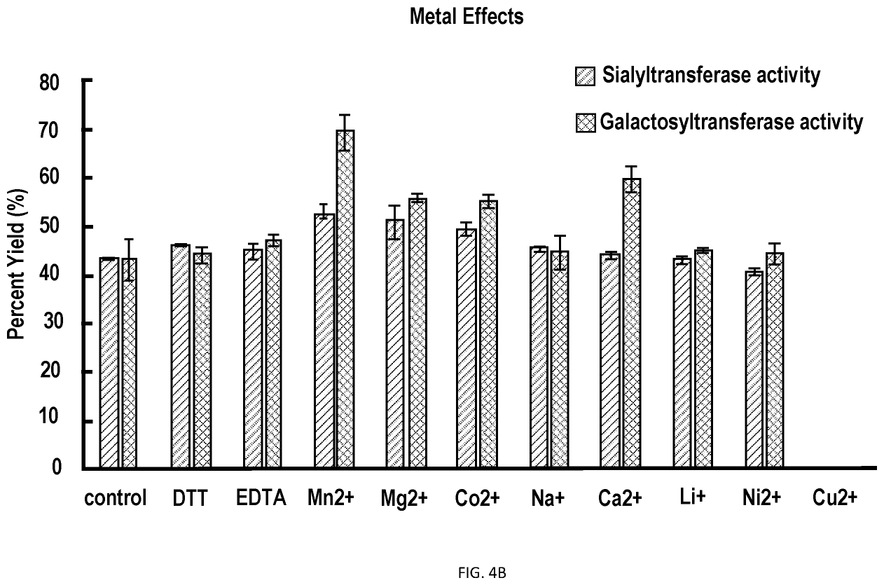

Using S1 as the acceptor substrate, the α1-4-galactosyltransferase activity of NmSiaD_{W} was shown to be active in a broad pH range of 5.0-9.5 and optimal activities were observed at pH 6.5 and pH 9.0. In comparison, using galactosyldisaccharide G2 (see below and FIG. 3) as the acceptor substrate, the α2-6-sialyltransferase activity of NmSiaD_{W} was shown to be active in a pH range of 6.0-9.5 with an optimum at pH 8.0 (FIG. 4A).

The addition of a metal ion such as Mn²⁺, Mg²⁺, Co²⁺, Na⁺, Ca²⁺, Li⁺, Ni²⁺ was not required and did not significantly affect either glycosyltransferase activities of NmSiaD_{W} although the addition of Cu²⁺ completely abolished both activities (FIG. 4B).

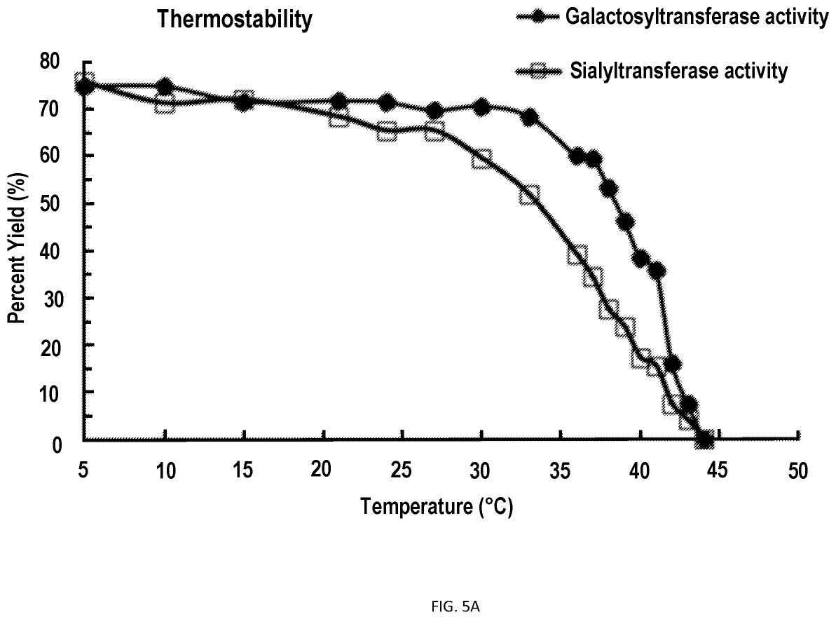

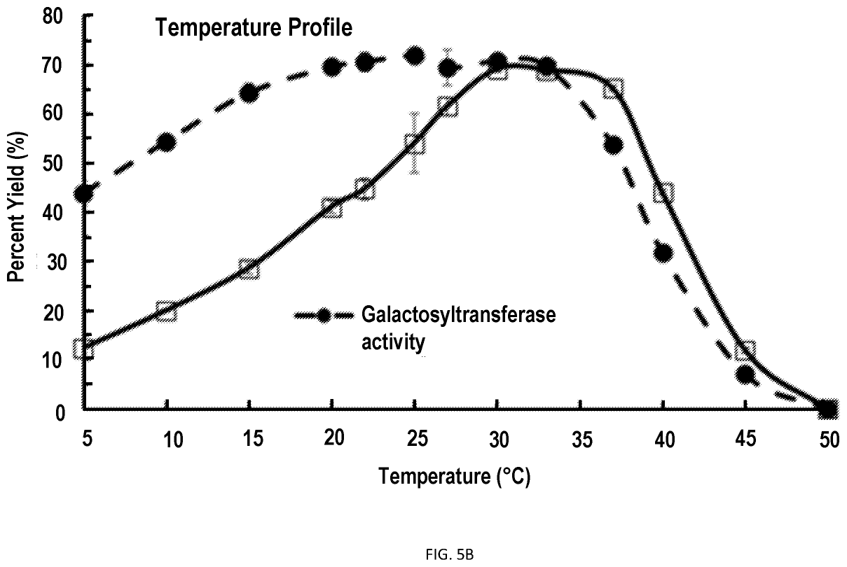

The α1-4-galactosyltransferase domain of NmSiaD_{W} seemed to be more stable than its a2-6-sialyltransferase domain. The activity of the former retained after being incubated for 30 minutes at a temperature up to 33° C., while the activity of the latter decreased significantly after incubation for 30 minutes at a temperature higher than 30° C. (FIG. 5A). Both glycosyltransferase activities were lost when NmSiaD_{W} was incubated at 44° C. for 30 minutes. The optimal temperature for the α1-4-galactosyltransferase activity was in a broad range of 20-33° C., while the optimal sialyltransferase activity had a narrower range of 30-37° C. (FIG. 5B). To retain the activity of other working enzymes, pH 8.0 and 20 mM MgCl₂ were determined for reactions using the one-pot multi-enzyme (OPME) system.

The OPME α1-4-galactosylation and α2-6-sialylation systems can be repeated in sequence using the newly obtained elongated oligosaccharides as acceptor substrates to obtain longer chain oligosaccharide products. Knowing the optimal conditions of both glycosyltransferase activities of NmSiaD_{W}, NmW CPS oligosaccharides ranging from galactosyldisaccharide G2 to galactosyldecasaccharide G10 were synthesized from sialylmonosaccharide Neu5AcαProNHCbz (S1) using a sequential one-pot multienzyme (OPME) process.

As shown in FIG. 3, an OPME α1-4-galactosylation system (OPME1) containing Streptococcus pneumoniae TIGR4 galactokinase (SpGalK), Bifidobacterium longum UDP-sugar pyrophosphorylase (BLUSP), Pasteurella multocida inorganic pyrophosphatase (PmPpA), and NmSiaD_{W} was used to add an α1-4-linked galactose residue to a sialoside acceptor such as S1. In this system, SpGalK was responsible for the formation of galactose-1-phosphate (Gal-1-P) which was used by BLUSP to form activated sugar nucleotide uridine-5′-diphosphate galactose (UDP-Gal), the donor substrate of the α1-4-galactosyltransferase activity of NmSiaD_{W} for the synthesis of galactosides such as G2. PmPpA was included to hydrolyze the inorganic pyrophosphate (PPi) formed in the BLUSP-catalyzed reaction to drive the reaction towards the formation of UDP-Gal.

All OPME reactions were carried out at 30° C. for 20 h in preparative-scale (200-450 mg), except monosaccharide reaction which took 7 days in total. The products were obtained in excellent yields after purification with a C18 reverse phase column twice. For the first round, the product was eluted by water containing 0.1% TFA and acetonitrile with a gradient. Protonated product was easily separated from polar reactants. Then the fractions containing products were collected and neutralized by NaOH to avoid oligosaccharides exposed to acidic conditions for a long time. Then the crude product was concentrated and purified by C18 column again. The product was eluted by water and acetonitrile with a gradient to remove salts from neutralization. Finally, the product was purified as a sodium salt. The structures and the purities of the products were confirmed by ¹H and ¹³C nuclear magnetic resonance (NMR) and high resolution mass spectrometry (HRMS). From S1, galactosyldisaccharide G2 (1.26 g) was synthesized and purified with an excellent 92% yield.

Subsequently, an OPME α2-6-sialylation system (OPME2) containing Neisseria meningitidis CMP-sialic acid synthetase (NmCSS) and NmSiaD_{W} was used to sialylate the galactoside formed. In this system, NmCSS catalyzed the formation of cytidine-5′-monophosphate Neu5Ac (CMP-Neu5Ac), the activated sugar nucleotide donor for the α2-6-sialyltransferase activity of NmSiaD_{W} for the synthesis of α2-6-linked sialosides such as S3 (FIG. 3). From G2, sialyltrisaccharide S3 (620 mg) was synthesized and purified with an excellent 96% yield.

Repeating the OPME α1-4-galactosylation and OPME α2-6-sialylation reactions sequentially with product purification after each OPME reaction to provide the acceptor substrate for the next OPME reaction led to the efficient synthesis of a series of NmW CPS oligosaccharides in 83-96% yields including G4 (556 mg, 91%), S5 (512 mg, 83%), G6 (440 mg, 88%), S7 (418 mg, 90%), G8 (380 mg, 96%), S9 (301 mg, 84%), and G10 (221 mg, 83%).

These reactions were carried out in 0.25-1.00 g scales in Tris-HCl buffer (100 mM, pH 8.0) containing MgCl₂ (20 mM) with the consideration of acceptable and optimal reaction conditions of NmSiaD_{W} and other enzymes involved in the sequential OPME reactions. Except for the synthesis of galactosyldisaccharide G2 from sialylmonosaccharide S1 which required a long reaction time (98 h), all other OPME reactions were carried out at 30° C. for 20 h. Assisted by the Cbz-tag, product purification was conveniently achieved by passing the reaction mixture through a C18 reverse phase column twice. The first column purification used a gradient solution of 0.1% trifluoroacetic acid (TFA) in H₂O and acetonitrile as an eluent to separate the protonated product from other components in the reaction mixture. The fractions containing the product were neutralized by NaOH immediately to minimize acid-catalyzed hydrolysis. The second C18 column purification used a gradient solution of water and acetonitrile to obtain the desired pure product whose structure and purity were confirmed by nuclear magnetic resonance (NMR), high resolution mass spectrometry (HRMS), and ultra-high performance liquid chromatography (UHPLC) analyses.

Heteronuclear Single Quantum Coherence-Total Correlation Spectroscopy (HSQC-TOCSY) studies for S1-G10 with 90 ms and 10 ms mixing times clearly show independent coupling networks of terminal and internal Neu5Ac or Gal residues. For example, for S3 which contains two Neu5Ac residues, the chemical shifts of the internal Neu5Ac are more downfield for H3_{eq}, H4, H5 H6 (0.05-0.20 ppm difference), and C4 (4.28 ppm difference) but more upfield for H3ₐₓ (0.09 ppm difference), C3 (3.32 ppm difference), and C5 (2.27 ppm difference) than those of the terminal Neu5Ac with no significant differences for C6 (data not shown). In comparison, for G4 which contains two Gal residues, the chemical shifts of the protons on the Gal backbones (less than 0.05 ppm difference) and C1 (0.65 ppm difference) are slightly more upfield for the internal residue (data not shown).

### Example 3. Synthesis and Characterization of Oligosaccharides

A. Chemical Synthesis of Acceptor Substrate Neu5AcαProNHCbz (S1)

Synthesis of Neu5Ac methyl ester (3). N-Acetylneuraminic acid (15.0 g, 0.49 mol) was suspended in dry methanol (200 mL) and Dowex 50WX4 (H⁺) resin (10 g) was added. The mixture was stirred at room temperature for overnight. The reaction was monitored by MS and TLC (EtOAc:MeOH:H₂O=4:2:1, by volume). Upon completion, the resin was removed by filtration, and the filtrate was concentrated in vacuo and dried under vacuum to yield 2 as a white solid. The obtained solid was dissolved in anhydrous pyridine (200 mL), followed by the addition of acetic anhydride (70 mL) and 4-dimethylaminopyridine (DMAP, 400 mg). The reaction was stirred at room temperature for overnight and the reaction was monitored by TLC (Hexane:EtOAc=1:3 by volume). The reaction mixture was diluted with 500 mL of ethyl acetate and extracted with water for three times. The organic layer was dried with anhydrous magnesium sulfate, filtered, and concentrated in vacuo. The product was purified by silica gel column (hexane:EtOAc=1:2 to 1:4, by volume) to obtain 22.2 g of the peracetylated product (3) with a yield of 86% for two steps.

Synthesis of Neu5AcαProNHCbz (S1). Peracetylated Neu5Ac methyl ester (3, 6.0 g, 11.3 mmol) was dissolved in anhydrous dichloromethane (20 mL) in a round bottom flask (200 mL) and the reaction was placed in an ice-water bath. Acetyl chloride (80 mL) was added followed by the addition of anhydrous methanol (2 mL) under Argon and the reaction mixture was stirred for 20 minutes. The reaction flask was then sealed and the mixture was stirred at room temperature for 2 days. The reaction progress was monitored by TLC analysis (hexane:EtOAc=1:4, by volume). Upon completion, the reaction mixture was concentrated, co-evaporated with toluene for three times, and dried under vacuum. Without further purification of the crude product, molecular sieves 4 Å (6.0 g), anhydrous dichloromethane (50 mL), and benzyl N-(3-hydroxypropyl) carbamate (4.78 g, 22.8 mmol) were added under argon. The mixture was placed in an ice-water bath and silver triflate (2.90 g, 11.3 mmol) was added. The reaction flask was covered with an aluminum foil and the mixture was stirred at room temperature for overnight. The reaction progress was monitored by TLC analysis (hexane:EtOAc=1:4, by volume). Upon completion, the reaction mixture was filtered over Celite and washed with DCM. The filtrate was concentrated for purification by silica gel column chromatography (hexane:EtOAc=1:1 to 1:4, by volume). Fractions were collected, concentrated, and dried under vacuum. (Note, when silver carbonate was used as a promoter instead of silver triflate, the glycosylation yield was much lower and glycal was formed as a major byproduct.)

The glycosylation product (5) was dissolved in anhydrous methanol (100 mL). Sodium methoxide was added until the pH was around 9.0, and the reaction mixture was stirred for overnight at r.t. The reaction was monitored by TLC analysis with two different developing solvent systems (EtOAc:hexane=4:1, by volume, to monitor consumption of starting material; and EtOAc:MeOH:H₂O=8:2:0.5, by volume, to monitor the formation of the deacetlyated product). Upon completion, the reaction mixture was neutralized by adding Dowex 50WX4 (H⁺) resin. The resin was then removed by filtration and the filtrate was concentrated in vacuo and dried under vacuum. The product was dissolved in 100 mL of a solvent mixture (water:methanol=4:1, by volume). The pH of the reaction mixture was adjusted to 9.0 using 2.0 M of NaOH and the mixture was stirred for overnight at r.t. The reaction was monitored by TLC analysis (EtOAc:MeOH:H₂O:AcOH=7:2:1:0.2, by volume). Up completion, the reaction mixture was neutralized by adding Dowex 50WX4 (H⁺) resin. The resin was then removed by filtration, and the filtrate was concentrated in vacuo. The crude product was purified by a silica gel column (EtOAc:MeOH:H₂O=8:2:1, by volume) to produce S1 (3.79 g, 67% for four steps).

¹H NMR (800 MHz, D₂O) δ 7.46-7.37 (m, 5H, Ar—H), 5.08 (s, 2H, O—CH₂—Ar), 3.87-3.75 (m, 4H, H-8, H-9, H-5, O—CH₂—CH₂), 3.70-3.64 (m, 2H, H-4, H-9), 3.61 (dd, J=11.9, 6.0 Hz, 1H, H-9), 3.59 (dt, J=9.1, 1.4 Hz, 1H, H-7), 3.50-3.45 (m, 1H, O—CH₂—CH₂), 3.23-3.11 (m, 2H, CH₂—NH), 2.72 (dd, J=12.5, 4.6 Hz, 1H, H-3eq), 2.03 (d, J=1.2 Hz, 3H, CH₃—CO), 1.73 (p, J=6.5 Hz, 2H, O—CH₂—CH₂—CH₂—NH), 1.65 (t, J=12.2 Hz, 1H, H-3). ¹³C NMR (200 MHz, D₂O) δ 175.06 (COOH), 173.66 (CH₃—CO), 158.33 (NH—COO), 136.52 (O—CH₂—Ar), 128.76 (Ar), 128.29 (Ar), 127.60 (Ar), 100.55 (C-2), 72.55 (C-6), 71.70 (C-8), 68.25 (C-4), 68.14 (C-7), 66.76 (O—CH₂—Ar), 62.50 (C-9), 62.07 (O—CH₂—CH₂), 51.90 (C-5), 40.35 (C-3), 37.52 (CH₂—NH), 28.92 (O—CH₂—CH₂—CH₂—NH), 22.01 (CH₃—CO). HRMS (ESI) m/z calculated for C₂₂H₃₁N₂O₁₁⁻ (M-H) 499.1928, found 499.1914.

B. One-Pot Four-Enzyme Preparative-Scale Synthesis of Acceptor Substrate Disaccharide Galα1-4Neu5AcαProNHCbz (G2).

A reaction mixture in Tris-HCl buffer (100 mM, pH 8.5) in a total volume of 200 mL containing Neu5AcαProNHCbz (S1) (1.00 g, 2.0 mmol), galactose (0.72 g, 4.0 mmol), ATP disodium salt (2.42 g, 4.4 mmol), UTP trisodium salt (2.42 g, 4.4 mmol), MgCl₂ (20 mM), SpGalK (22 mg), BLUSP (22 mg), PmPpA (22 mg), and NmSiaD_{W} (22 mg) was incubated in a 250-mL bottle in a shaker (100 rpm) at 30° C. for 98 hrs. The reaction progress was monitored by UHPLC (AdvanceBio Glycan Map, Agilent, 87% Acetonitrile+0.1% TFA in water, monitored at 215 nm). When an optimal yield was achieved, pre-chilled ethanol (200 mL) was added and the resulting mixture was incubated at 4° C. for 30 min. The precipitates were removed by centrifugation (4300×g, 30 min, 4° C.). The supernatant was concentrated and purified by a C18 column in a CombiFlash Rf 200i system with a gradient of water with 0.1% TFA (v/v) and acetonitrile (0-100% acetonitrile) for elution. Fractions containing the product were collected, neutralized, concentrated, and further purified by a C18 column to produce disaccharide Galα1-4Neu5AcαProNHCbz (G2) as a sodium salt (1.26 g, 92%).

¹H NMR (800 MHz, D₂O) δ 7.46-7.37 (m, 5H, Ar—H), 5.09 (s, 2H, O—CH₂—Ar), 5.08 (d, J=4.0 Hz, 1H, H″-1), 4.04 (t, J=10.3 Hz, 1H, H′-5), 3.96 (d, J=3.3 Hz, 1H, H″-3), 3.86 (td, J=6.3, 3.1 Hz, 1H, H′-8), 3.84-3.77 (m, 5H, H′-5, H′-9, H″-2, H″-5, O—CH₂—CH₂), 3.75-3.68 (m, 4H, H′-4, H″-4, H″-6), 3.64-3.59 (m, 2H, H′-7, H′-9), 3.52-3.47 (m, 1H, O—CH₂—CH₂), 3.23-3.14 (m, 2H, CH₂—NH), 2.88 (dd, J=12.5, 4.7 Hz, 1H, H′-3_{eq}), 2.03 (d, J=1.6 Hz, 3H, CH₃—CO), 1.74 (p, J=6.6 Hz, 2H, O—CH₂—CH₂—CH₂—NH), 1.62 (t, J=12.0 Hz, 1H, H′-3ax).

¹³C NMR (200 MHz, D₂O) δ 174.32 (COOH), 173.49 (CH₃—CO), 158.34 (NH—COO), 136.53 (O—CH₂—Ar), 128.76 (Ar), 128.28 (Ar), 127.58 (Ar), 100.60 (C′-2), 94.72 (C″-1), 72.82 (C′-4), 72.19 (C′-6), 71.77 (C′-8), 71.02 (C″-5), 69.36 (C″-4), 69.05 (C″-3), 68.04 (C′-7), 67.88 (C″-2), 66.76 (O—CH₂—Ar), 62.48 (C′-9), 62.07 (O—CH₂—CH₂), 60.73 (C″-6), 49.53 (C′-5), 37.47 (CH₂—NH), 36.73 (C′-3), 28.91 (O—CH₂—CH₂—CH₂—NH), 22.11 (CH₃—CO). HRMS (ESI) m/z calculated for C₂₈H₄₁N₂O₁₆⁻ (M-H) 661.2456, found 661.2458.

One-pot two-enzyme preparative-scale synthesis of trisaccharide Neu5Acα2-6Galα1-4Neu5AcαProNHCbz (S3). A reaction mixture in Tris-HCl buffer (100 mM, pH 8.5) in a total volume of 64 mL containing galactosyldisaccharide G2 (426 mg, 0.64 mmol), Neu5Ac (260 mg, 0.83 mmol), CTP disodium salt (445 mg, 0.83 mmol), MgCl₂ (20 mM), NmCSS (4 mg), and NmSiaD_{W} (4 mg) was incubated in a 250-mL bottle in a shaker (100 rpm) at 30° C. for 15 hrs. The reaction progress was monitored by UHPLC (AdvanceBio Glycan Map, Agilent, 87% Acetonitrile+0.1% TFA in water, monitored at 215 nm). When an optimal yield was achieved, pre-chilled ethanol (64 mL) was added and the resulting mixture was incubated at 4° C. for 30 min. Procedures for centrifugation, concentration, purification, collection and neutralization were similar to that described above for G2 to produce sialyltrisaccharide S3 as a sodium salt (620 mg, 96%).

¹H NMR (800 MHz, D₂O) δ 7.49-7.35 (m, 5H, Ar—H), 5.11 (s, 2H, O—CH₂—Ar), 5.06 (d, J=3.9 Hz, 1H, H″-1), 4.03 (t, J=10.3 Hz, 1H, H′-5), 3.96 (d, J=3.4 Hz, 1H, H″-3), 3.90-3.74 (m, 10H), 3.73-3.59 (m, 8H), 3.56 (dd, J=9.0, 1.7 Hz, 1H), 3.50 (dt, J=10.6, 6.1 Hz, 1H, O—CH₂—CH₂), 3.24-3.15 (m, 2H, CH₂—NH), 2.88 (dd, J=12.5, 4.7 Hz, 1H, H′-3_{eq}), 2.73 (dd, J=12.4, 4.7 Hz, 1H, H′″-3_{eq}), 2.07 (s, 3H, H′—CH₃—CO), 2.03 (s, 3H, H′″—CH₃—CO), 1.74 (p, J=6.8 Hz, 2H, O—CH₂—CH₂—CH₂—NH), 1.70 (t, J=12.2 Hz, 1H, H′″-3ₐₓ), 1.61 (t, J=12.0 Hz, 1H, H′-3ax).

¹³C NMR (200 MHz, D₂O) δ 174.97, 174.49, 173.48, 173.32, 158.37 (NH—COO), 136.56 (O—CH₂—Ar), 128.75 (Ar), 128.27 (Ar), 127.56 (Ar), 100.59 (C′-2), 100.11 (C′″-2), 94.54 (C″-1), 72.69 (C′-4), 72.46 (C′″-6), 72.09, 71.83, 69.52, 69.19, 68.89 (C″-3), 68.37 (C′″-4), 68.27, 68.03, 67.84 (C″-2), 66.76 (O—CH₂—Ar), 62.62, 62.60, 62.52 (C″-6), 62.06 (O—CH₂—CH₂), 51.79 (C′″-5), 49.52 (C′-5), 40.06 (C′″-3), 37.47 (CH₂—NH), 36.64 (C′-3), 28.89 (O—CH₂—CH₂—CH₂—NH), 22.42 (H′—CH₃—CO), 22.00 (H′″—CH₃—CO). HRMS (ESI) m/z calculated for C₃₉H₅₈N₃O₂₄⁻ (M-H) 952.3410, found 952.3390.

One-pot four-enzyme preparative-scale synthesis of galactosyltetrasaccharide Galα1-4Neu5Acα2-6Galα1-4Neu5AcαProNHCbz (G4). A reaction mixture in Tris-HCl buffer (100 mM, pH 8.5) in a total volume of 53 mL containing sialyltrisaccharide S3 (528 mg, 0.53 mmol), galactose (124 mg, 0.67 mmol), ATP disodium salt (380 mg, 0.67 mmol), UTP trisodium salt (379 mg, 0.67 mmol), MgCl₂ (20 mM), SpGalK (4.8 mg), BLUSP (4.8 mg), PmPpA (4.8 mg) and NmSiaD_{W} (2.4 mg) was incubated in a 125-mL bottle in a shaker (100 rpm) at 30° C. for 16 hrs. The reaction progress was monitored by UHPLC (AdvanceBio Glycan Map, Agilent, 80% Acetonitrile+0.1% TFA in water, monitored at 215 nm). When an optimal yield was achieved, pre-chilled ethanol (53 mL) was added and the resulting mixture was incubated at 4° C. for 30 min. Procedures for centrifugation, concentration, purification, collection and neutralization were similar to that described above for G2 to produce galactosyltetrasaccharide G4 as a sodium salt (556 mg, 91%).

¹H NMR (800 MHz, D₂O) δ 7.49-7.37 (m, 5H, Ar—H), 5.10 (s, 2H, O—CH₂—Ar), 5.08 (d, J=4.0 Hz, 1H, H″″-1), 5.06 (d, J=3.9 Hz, 1H, H″-1), 4.04 (td, J=10.3, 5.9 Hz, 2H, H′-5, H′″-5), 3.96 (dd, J=10.1, 3.4 Hz, 2H, H″-3, H″″-3), 3.90-3.59 (m, 23H), 3.50 (dt, J=10.9, 6.2 Hz, 1H, O—CH₂—CH₂), 3.23-3.15 (m, 2H, CH₂—NH), 2.88 (td, J=12.1, 4.6 Hz, 2H, H′-3eq, H′″-3_{eq}), 2.08 (s, 3H, H′—CH₃—CO), 2.03 (s, 3H, H′″—CH₃—CO), 1.75 (h, J=7.4, 6.9 Hz, 2H, O—CH₂—CH₂—CH₂—NH), 1.66 (t, J=12.1 Hz, 1H, H′″-3ₐₓ), 1.61 (t, J=12.0 Hz, 1H, H′-3ₐₓ).

¹³C NMR (200 MHz, D₂O) δ 174.49, 174.30, 173.34, 173.18, 158.36 (NH—COO), 136.57 (O—CH₂—Ar), 128.76 (Ar), 128.27 (Ar), 127.56 (Ar), 100.62 (C′-2), 100.23 (C′″-2), 95.05 (C″″-1), 94.40 (C″-1), 73.23, 72.49, 72.19, 72.09, 71.91, 71.79, 71.02, 69.56, 69.23, 69.21, 69.04 (C″″-3), 68.94 (C″-3), 68.17, 67.96, 67.86, 67.80, 66.76 (O—CH₂—Ar), 62.86, 62.60, 62.46, 62.06 (O—CH₂—CH₂), 60.72 (C″″-4), 49.54 (C′-5), 49.46 (C′″-5), 37.48 (CH₂—NH), 36.77 (C′″-3), 36.57 (C′-3), 28.90 (O—CH₂—CH₂—CH₂—NH), 22.43 (H′—CH₃—CO), 22.11 (H′″—CH₃—CO). HRMS (ESI) m/z calculated for C₄₅H₆₈N₃O₂₉ (M-H) 1114.3939, found 1114.3918.

One-pot two-enzyme preparative-scale synthesis of sialylpentasaccharide Neu5Acα2(-6Galα1-4Neu5Ac(×2)₂-ProNHCbz (S5). A reaction mixture in Tris-HCl buffer (100 mM, pH 8.5) in a total volume of 43 mL containing galactosyltetrasaccharide G4 (502 mg, 0.43 mmol), Neu5Ac (175 mg, 0.56 mmol), CTP disodium salt (300 mg, 0.56 mmol), MgCl₂ (20 mM), NmCSS (1.0 mg) and NmSiaD_{W} (2.0 mg) was incubated in a 125-mL bottle in a shaker (100 rpm) at 30° C. for 17 hrs. The reaction progress was monitored by UHPLC (AdvanceBio Glycan Map, Agilent, 75% Acetonitrile+0.1% TFA in water, monitored at 215 nm). When an optimal yield was achieved, pre-chilled ethanol (43 mL) was added and the resulting mixture was incubated at 4° C. for 30 min. Procedures for centrifugation, concentration, purification, collection and neutralization were similar to that described above for G2 to produce sialylpentasaccharide S5 as a sodium salt (512 mg, 83%).

¹H NMR (800 MHz, D₂O) δ 7.46-7.37 (m, 5H, Ar—H), 5.10 (s, 2H, O—CH₂—Ar), 5.06 (d, J=3.7 Hz, 2H, H^{II,IV}-1), 4.03 (td, J=10.2, 5.7 Hz, 2H, H^{I,III}-5), 3.96 (d, J=3.4 Hz, 2H, H^{II,IV}-3), 3.92-3.59 (m, 29H), 3.58-3.54 (m, 1H), 3.50 (dt, J=11.6, 6.2 Hz, 1H, O—CH₂—CH₂), 3.24-3.17 (m, 2H, CH₂—NH), 2.88 (dt, J=12.5, 4.4 Hz, 2H, H^{I,III}-3eq), 2.72 (dd, J=12.4, 4.7 Hz, 1H, H^{V}-3eq), 2.08 (s, 6H, H^{I,III}—CH₃—CO), 2.03 (s, 3H, H^{V}—CH₃—CO), 1.74 (p, J=6.8 Hz, 2H, O—CH₂—CH₂—CH₂—NH), 1.70 (t, J=12.2 Hz, 1H, H^{V}-3ax), 1.63 (dt, J=30.6, 12.0 Hz, 2H, H^{I,III}-3ₐₓ).

¹³C NMR (200 MHz, D₂O) δ 174.98, 174.51, 174.48, 173.49, 173.39, 173.06, 158.37, 136.56, 128.77, 128.28, 127.57, 100.58, 100.22, 100.14, 94.88, 94.61, 73.11, 72.76, 72.45, 72.11, 72.08, 72.04, 72.01, 71.84, 71.82, 69.59, 69.56, 69.19, 69.08, 68.91, 68.90, 68.38, 68.29, 68.18, 67.98, 67.85, 67.78, 66.76, 62.76, 62.66, 62.64, 62.63, 62.50, 62.46, 62.06, 51.80, 49.56, 49.47, 40.04, 37.48, 36.67, 28.91, 22.48, 22.02. HRMS (ESI) m/z calculated for C₅₆H₈₅N₄O₃₇⁻ (M-H) 1405.4893, found 1405.4896.

One-pot four-enzyme preparative-scale synthesis of galactosylhexasaccharide Galα1(-4Neu5Acα2-6Galα1)₂-4Neu5AcαProNHCbz (G6). A reaction mixture in Tris-HCl buffer (100 mM, pH 8.5) in a total volume of 31 mL containing sialylpentasaccharide S5 (450 mg, 0.31 mmol), galactose (73 mg, 0.40 mmol), ATP disodium salt (222 mg, 0.40 mmol), UTP trisodium salt (220 mg, 0.40 mmol), MgCl₂ (20 mM), SpGalK (2.4 mg), BLUSP (2.4 mg), PmPpA (2.4 mg), and NmSiaD_{W} (1.2 mg) was incubated in a bottle (125 mL) in a shaker (100 rpm) at 30° C. for 16 hrs. The product formation was monitored by UHPLC (AdvanceBio Glycan Map, Agilent, 75% Acetonitrile+0.1% TFA in water, monitored at 215 nm). When an optimal yield was achieved, pre-chilled ethanol (31 mL) was added and the resulting mixture was incubated at 4° C. for 30 min. Procedures for centrifugation, concentration, purification, collection and neutralization were similar to that described above for G2 to produce galactosylhexasaccharide G6 as a sodium salt (440 mg, 88%).

¹H NMR (800 MHz, D₂O) δ 7.53-7.29 (m, 5H, Ar—H), 5.11 (s, 2H, O—CH₂—Ar), 5.08 (d, J=4.0 Hz, 1H, H^{VI}-1), 5.05 (d, J=3.9 Hz, 2H, H^{II,IV}-1), 4.03 (ddt, J=10.3, 6.6, 3.3 Hz, 3H, H^{I,III,V}-5), 3.98-3.94 (m, 3H, H^{II,IV,VI}-3), 3.91-3.57 (m, 34H), 3.50 (dt, J=11.1, 6.3 Hz, 1H, O—CH₂—CH₂), 3.24-3.16 (m, 2H, CH₂—NH), 2.87 (ddd, J=12.4, 7.8, 4.8 Hz, 3H, H^{I,III,V}-3eq), 2.08 (s, 6H, H^{I,III}—CH₃—CO), 2.02 (s, 3H, H^{V}—CH₃—CO), 1.74 (p, J=6.9 Hz, 2H, O—CH₂—CH₂—CH₂—NH), 1.69-1.58 (m, 3H, H^{I,III,V}-3ₐₓ).

¹³C NMR (200 MHz, D₂O) δ 174.52, 174.47, 174.29, 173.38, 173.20, 173.08, 158.37, 136.57, 128.76, 128.27, 127.56, 100.57, 100.22, 95.04, 94.79, 94.64, 73.23, 72.99, 72.79, 72.16, 72.10, 72.07, 71.94, 71.88, 71.82, 71.01, 69.59, 69.23, 69.18, 69.09, 69.05, 68.97, 68.89, 68.19, 68.11, 67.98, 67.84, 67.81, 67.78, 66.76, 62.90, 62.77, 62.60, 62.58, 62.51, 62.06, 60.71, 49.55, 49.47, 49.46, 37.48, 36.74, 36.69, 36.63, 28.90, 22.47, 22.11. HRMS (ESI) m/z calculated for C₆₂H₉₅N₄O₄₂ (M-H) 1567.5421, found 1567.5397.

One-pot two-enzyme preparative-scale synthesis of sialylheptasaccharide Neu5Acα2(-6Galα1-4Neu5Acα2)₃-ProNHCbz (S7). A reaction mixture in Tris-HCl buffer (100 mM, pH 8.5) in a total volume of 25 mL containing galactosylhexasaccharide G6 (390 mg, 0.25 mmol), Neu5Ac (102 mg, 0.33 mmol), CTP disodium salt (175 mg, 0.33 mmol), MgCl₂ (20 mM), NmCSS (0.8 mg), and NmSiaD_{W} (1.6 mg) was incubated in a 50-mL centrifuge tube in a shaker (100 rpm) at 30° C. for 16 hrs. The product formation was monitored by UHPLC (AdvanceBio Glycan Map, Agilent, 72% Acetonitrile+0.1% TFA in water, monitored at 215 nm). When an optimal yield was achieved, pre-chilled ethanol (25 mL) was added and the resulting mixture was incubated at 4° C. for 30 min. Procedures for centrifugation, concentration, purification, collection and neutralization were similar to that described above for G2 to produce sialylheptasaccharide S7 as a sodium salt (418 mg, 90%).

¹H NMR (800 MHz, D₂O) δ 7.49-7.36 (m, 5H, Ar—H), 5.11 (s, 2H, O—CH₂—Ar), 5.05 (q, J=3.7 Hz, 3H, H^{II,IV,VI}-1), 4.03 (td, J=10.3, 3.6 Hz, 3H, H_{I,III,V}-5), 3.96 (d, J=3.4 Hz, 3H, H^{II,IV,VI}-3), 3.91-3.60 (m, 39H), 3.56 (dd, J=11.9, 3.2 Hz, 2H), 3.50 (dt, J=10.7, 6.2 Hz, 1H, O—CH₂—CH₂), 3.23-3.16 (m, 2H, CH₂—NH), 2.87 (dt, J=12.7, 5.6 Hz, 3H, H^{I,III,V}-3eq), 2.72 (dd, J=12.4, 4.7 Hz, 1H, H^{VII}-3eq), 2.08 (s, 9H, H^{I,III,V}—CH₃—CO), 2.03 (s, 3H, H^{VII}—CH₃—CO), 1.74 (p, J=7.0 Hz, 2H, O—CH₂—CH₂—CH₂—NH), 1.70 (t, J=12.2 Hz, 1H, H^{VII}-3ₐₓ), 1.63 (dt, J=33.5, 12.0 Hz, 3H, H^{I,III,V}-3ₐₓ).

¹³C NMR (200 MHz, D₂O) δ 174.96, 174.52, 174.49, 174.46, 173.48, 173.09, 173.05, 158.37, 136.57, 128.76, 128.27, 127.56, 100.57, 100.23, 100.21, 100.13, 95.07, 94.88, 94.67, 73.33, 73.11, 72.81, 72.44, 72.11, 72.08, 72.05, 71.96, 71.95, 71.82, 69.62, 69.59, 69.55, 69.17, 69.09, 69.03, 68.91, 68.39, 68.28, 68.19, 68.12, 67.97, 67.84, 67.80, 67.76, 66.76, 62.80, 62.65, 62.63, 62.61, 62.51, 62.06, 51.80, 49.56, 49.48, 40.04, 37.47, 36.77, 36.71, 36.64, 28.89, 22.51, 22.47, 22.00. HRMS (ESI) m/z calculated for C₇₃H₁₁₂N₅O₅₀⁻ (M-H) 1858.6375, found 1858.6323.

One-pot four-enzyme preparative-scale synthesis of galactosyloctasaccharide Galα1(-4Neu5Acα2-6Galα1)₃-4Neu5AcαProNHCbz (G8). A reaction mixture in Tris-HCl buffer (100 mM, pH 8.5) in a total volume of 19 mL containing sialylheptasaccharide S7 (370 mg, 0.19 mmol), galactose (47 mg, 0.25 mmol), ATP disodium salt (143 mg, 0.25 mmol), UTP trisodium salt (143 mg, 0.25 mmol), MgCl₂ (20 mM), SpGalK (1.8 mg), BLUSP (1.8 mg), PmPpA (1.8 mg), and NmSiaD_{W} (0.9 mg) was incubated in a 50-mL centrifuge tube in a shaker (100 rpm) at 30° C. for 16 hrs. The product formation was monitored by UHPLC (AdvanceBio Glycan Map, Agilent, 72% Acetonitrile+0.1% TFA in water, monitored at 215 nm). When an optimal yield was achieved, pre-chilled ethanol (19 mL) was added and the resulting mixture was incubated at 4° C. for 30 min. Procedures for centrifugation, concentration, purification, collection and neutralization were similar to that described above for G2 to produce galactosyloctasaccharide G8 as a sodium salt (380 mg, 95%).

¹H NMR (800 MHz, D₂O) δ 7.46-7.37 (m, 5H, Ar—H), 5.11 (s, 2H, O—Ar), 5.07 (d, J=4.0 Hz, 1H, H^{VIII}-1), 5.05 (dd, J=6.8, 4.0 Hz, 3H, H^{II,IV,VI}-1), 4.06-4.00 (m, 4H, H^{I,III,V,VII}-5), 3.96 (t, J=3.9 Hz, 4H, H^{II,IV,VI,VIII}-3), 3.90-3.58 (m, 45H), 3.49 (dd, J=10.6, 5.7 Hz, 1H, O—CH₂—CH₂), 3.23-3.17 (m, 2H, CH₂—NH), 2.90-2.84 (m, 4H, H^{I,III,V,VII}-3eq), 2.08 (s, 9H, H^{I,III,V}—CH₃—CO), 2.02 (s, 3H, H^{VII}—CH₃—CO), 1.74 (p, J=6.8 Hz, 2H, O—CH₂—CH₂—CH₂—NH), 1.68-1.58 (m, 4H, H^{I,III,V,VII}-3ₐₓ).

¹³C NMR (200 MHz, D₂O) δ 174.52, 174.50, 174.46, 174.29, 173.20, 173.08, 158.37, 136.57, 128.76, 128.27, 127.56, 100.57, 100.23, 100.21, 95.08, 95.05, 94.77, 94.68, 73.33, 73.24, 72.97, 72.81, 72.16, 72.11, 72.08, 72.05, 71.95, 71.91, 71.88, 71.82, 71.01, 69.62, 69.59, 69.23, 69.17, 69.11, 69.05, 69.03, 68.97, 68.90, 68.19, 68.13, 67.97, 67.84, 67.81, 67.76, 66.75, 62.89, 62.80, 62.61, 62.59, 62.51, 62.06, 60.71, 49.56, 49.48, 49.46, 37.47, 36.78, 36.74, 36.72, 36.71, 36.59, 28.89, 22.51, 22.47, 22.11. HRMS (ESI) m/z calculated for C₇₉H₁₂₁N₅O₅₅²⁻ (M/2-H) 1009.8413, found 1009.8401.

One-pot two-enzyme preparative-scale synthesis of sialylnonasaccharide Neu5Acα2(-6Galα1-4Neu5Acα2)₄-ProNHCbz (S9). A reaction mixture in Tris-HCl buffer (100 mM, pH 8.5) in a total volume of 15 mL containing galactosyloctasaccharide G8 (310 mg, 0.15 mmol), Neu5Ac (61 mg, 0.20 mmol), CTP disodium salt (105 mg, 0.20 mmol), MgCl₂ (20 mM), NmCSS (0.5 mg) and NmSiaD_{W} (1.0 mg) was incubated in a 50-mL centrifuge tube in a shaker (100 rpm) at 30° C. for 16 hrs. The product formation was monitored by UHPLC (AdvanceBio Glycan Map, Agilent, 71% Acetonitrile+0.1% TFA in water, monitored at 215 nm). When an optimal yield was achieved, pre-chilled ethanol (15 mL) was added and the resulting mixture was incubated at 4° C. for 30 min. Procedures for centrifugation, concentration, purification, collection and neutralization were similar to that described above for G2 to produce sialylnonasaccharide S9 as a sodium salt (301 mg, 84%).

¹H NMR (800 MHz, D₂O) δ 7.46-7.38 (m, 5H, Ar—H), 5.11 (s, 2H, O—CH₂—Ar), 5.05 (q, J=3.9 Hz, 4H, H^{II,IV,VI,VIII}-1), 4.03 (td, J=10.3, 4.2 Hz, 4H, H^{I,III,V,VII}-5), 3.96 (d, J=3.5 Hz, 4H, H^{II,IV,VI,VIII}-3), 3.92-3.75 (m, 28H), 3.73-3.59 (m, 23H), 3.56 (dd, J=8.8, 1.8 Hz, 1H), 3.50 (dt, J=11.1, 6.4 Hz, 1H, O—CH₂—CH₂), 3.23-3.14 (m, 2H, CH₂—NH), 2.90-2.84 (m, 4H, H^{I,III,V,VII}-3eq), 2.72 (dd, J=12.5, 4.6 Hz, 1H, H^{IX}-3eq), 2.08 (s, 12H, H^{I,III,V,VII}—CH₃—CO), 2.03 (s, 3H, H^{IX}—CH₃—CO), 1.74 (p, J=6.9 Hz, 2H, O—CH₂—CH₂—CH₂—NH), 1.65 (ddt, J=36.8, 33.3, 12.1 Hz, 5H, H^{I,III,VI,IX}-3ax).

¹³C NMR (200 MHz, D₂O) δ 174.96, 174.53, 174.51, 174.48, 174.46, 173.48, 173.09, 173.06, 158.37, 136.57, 128.76, 128.27, 127.56, 100.57, 100.23, 100.21, 100.13, 95.09, 95.04, 94.87, 94.68, 73.34, 73.30, 73.10, 72.81, 72.45, 72.11, 72.11, 72.06, 71.97, 71.95, 71.92, 71.82, 69.62, 69.62, 69.59, 69.55, 69.17, 69.09, 69.05, 69.03, 68.91, 68.39, 68.29, 68.19, 68.14, 68.12, 67.97, 67.84, 67.79, 67.78, 67.76, 66.76, 62.84, 62.79, 62.61, 62.51, 62.06, 51.80, 49.56, 49.48, 40.04, 37.47, 36.78, 36.71, 36.64, 28.89, 23.23, 22.51, 22.47, 22.01. HRMS (ESI) m/z calculated for C₉₀H₁₃₈N₆O₆₃^{2- (M/}2-H) 1155.3890, found 1155.3855.

One-pot four-enzyme preparative-scale synthesis of galactosyldecasaccharide Galα1(-4Neu5Acα2-6Galα1)₄-4Neu5AcαProNHCbz (G10). A reaction mixture in Tris-HCl buffer (100 mM, pH 8.5) in a total volume of 10 mL containing sialylnonasaccharide S9 (250 mg, 0.10 mmol), galactose (25 mg, 0.13 mmol), ATP disodium salt (75 mg, 0.13 mmol), UTP trisodium salt (250 mg, 0.13 mmol), MgCl₂ (20 mM), SpGalK (1.0 mg), BLUSP (1.0 mg), PmPpA (1.0 mg), and NmSiaD_{W} (0.5 mg) was incubated in a 50-mL centrifuge tube in a shaker (100 rpm) at 30° C. for 16 hrs. The product formation was monitored by UHPLC (AdvanceBio Glycan Map, Agilent, 70% acetonitrile+0.1% TFA in water, monitored at 215 nm). When an optimal yield was achieved, pre-chilled ethanol (10 mL) was added and the resulting mixture was incubated at 4° C. for 30 min. Procedures for centrifugation, concentration, purification, collection and neutralization were similar to that described above for G2 to produce galactosyldecasaccharide G10 as a sodium salt (221 mg, 83%).

¹H NMR (800 MHz, D₂O) δ 7.54-7.35 (m, 5H, Ar—H), 5.11-5.04 (m, 7H, O—CH₂—Ar, H^{II,IV,VII,VIII,X}-1), 4.05 (t, J=10.1 Hz, 5H, H^{I,III,V,VII,IX}-5), 3.95 (dd, J=6.2, 3.4 Hz, 5H, H^{II,IV,VI,VIII,X}-3), 3.91-3.58 (m, 56H), 3.55-3.50 (m, 1H, O—CH₂—CH₂), 3.24-3.13 (m, 2H, CH₂—NH), 2.95-2.78 (m, 5H, H^{I,III,V,VII,IX}-3eq), 2.06 (s, 12H, H^{I,III,V,VII}—CH₃—CO), 2.02 (s, 3H, H^{IX}—CH₃—CO), 1.77-1.71 (m, 2H, O—CH₂—CH₂—CH₂—NH), 1.71-1.60 (m, 5H, H^{I,III,V,VII,IX}-3ₐₓ).

¹³C NMR (200 MHz, D₂O) δ 174.46, 174.35, 171.70, 171.24, 171.16, 171.13, 158.34, 136.54, 128.75, 128.29, 127.57, 99.17, 94.73, 94.41, 94.36, 94.28, 72.37, 72.24, 71.88, 71.75, 71.70, 71.17, 71.05, 71.00, 70.98, 70.93, 69.54, 69.52, 69.31, 69.24, 69.22, 69.02, 68.13, 68.11, 68.04, 67.87, 67.79, 67.77, 67.76, 66.76, 63.21, 62.88, 62.84, 62.81, 62.06, 60.72, 49.34, 49.28, 37.36, 35.99, 35.54, 28.77, 22.33, 22.32, 22.11. HRMS (ESI) m/z calculated for C₉₆H₁₄₈N₆O₆₈²⁻ (M/2-H) 1236.4153, found 1236.4109.

### Example 4. Study of NmSiaD_{W} Donor Specificity

A. Experimental Procedure

Galactosyltransferase activity was assayed in reaction buffer (100 mM MES, pH 6.5, 10 mM MgCl₂) in the presence of 2 mM UDP-sugars and 1 mM Neu5Acα2-6Galα1-4Neu5AcαProNHCbz in a total volume of 10 μL. Reaction was performed at 30° C. with 0.11 μg NmSiaD_{W} for 10 min or 6.6 μg NmSiaD_{W} for 10 h. Reaction was quenched by addition of 10 μL pre-chilled ethanol and incubated at −20° C. for 30 min. UDP-sugar used were UDP-Gal, UDP-Glc, UDP-GalNAc, UDP-GlcNAc, UDP-Mannose, UDP-ManNAc, UDP-GalA and UDP-GlcA. GDP-Fuc and CMP-Neu5Ac were also included.

Sialyltransferase activity was assayed with one-pot multi-enzyme reactions. One-pot three-enzyme reactions were carried out in reaction buffer (100 mM Tris-HCl, pH 8.5) in the presence of 1.2 mM sialic acid precursors, 5 mM sodium pyruvate, 1.2 mM CTP and 1 mM Galα1-4Neu5Acα2-6Galα1-4Neu5AcαProNHCbz. 5.0 μg PmAldolase and 4.8 μg NmCSS were included in a total volume of 10 μL. Reaction was performed at 30° C. with 0.13 μg NmSiaD_{W} for 10 min or 7.8 μg NmSiaD_{W} for 10 h. Reaction was quenched by addition of 10 μL pre-chilled ethanol and incubated at −20° C. for 30 min. Sialic acid precursors used were mannose, ManNAc6N₃, ManNAc4N₃, ManNAc6OMe, ManNAz, ManNAc6NAc and ManNAc6F.

One-pot two-enzyme reactions were carried out in reaction buffer (100 mM Tris-HCl, pH 8.5) in the presence of 1.2 mM sialic acid or its derivatives, 1.2 mM CTP and 1 mM Galα1-4Neu5Acα2-6Galα1-4Neu5AcαProNHCbz. 4.8 μg NmCSS was included in a total volume of 10 μL. Reaction was performed at 30° C. with 0.13 μg NmSiaD_{W} for 10 min or 7.8 μg NmSiaD_{W} for 10 h. Reaction was quenched by addition of 10 μL pre-chilled ethanol and incubated at −20° C. for 30 min. Sialic acids and their derivatives used were Neu5Ac, Neu5Gc, Neu5GcOMe, Neu5Ac8OMe, Neu5,9Ac2 and Neu5,9Ac₂.

Samples were analyzed using UPLC with EcilpsePlusC18 column or AdvancBio Glycan Map column, Agilent. The samples were further analyzed by electrospray ionization (ESI)-HRMS using a Thermo Electron LTQ-Orbitrap Hybrid MS in a negative mode.

B. Results

Using sialyltrisaccharide S3 as the acceptor substrate, eight UDP-sugars as well as GDP-fucose and CMP-Neu5Ac were tested as potential donor substrates for the α1-4-galactosyltransferase activity of NmSiaD_{W}. As shown in Table 1, compared to UDP-Gal which is the native donor substrate, UDP-Glc is a weaker donor substrate. Quite interestingly, UDP-GalNAc was shown to be tolerated as poor donor substrate as well. Other sugar nucleotides tested were not tolerated.

TABLE 1
Donor substrate specificity for the αl-4-galactosyltransferase
activity of NmSiaD_{W} using different sugar nucleotides.

| | | Percentage conversion (%) | |
|---|---|---|---|
| | | 11 μg/mL | 0.66 mg/mL |
| | | NmSiaD_{W}, | NmSiaD_{W}, |
| | Substrates | 10 min | 10 h |
| 1 | UDP-Gal | 19.6 ± 0.4 | 100 |
| 2 | UDP-Glc | 0 | 11.6 ± 1.5 |
| 3 | UDP-GalNAc | 0 | 1.9 ± 0.2 |
| 4 | UDP-GlcNAc | 0 | 0 |
| 5 | UDP-GalA | 0 | <1 |
| 6 | UDP-GlcA | 0 | 0 |
| 7 | UDP-Mannose | 0 | 0 |
| 8 | UDP-ManNAc | 0 | 0 |
| 9 | GDP-Fucose | 0 | 0 |
| 10 | CMP-Neu5Ac | 0 | 0 |

Abbreviations: UDP, uridine 5′-diphosphate; Gal, galactose; Glc, glucose, GalNAc, N-acetylgalactosamine; GlcNAc, N-acetylglucosamine; GalA, galacturonic acid; GlcA, glucuronic acid; ManNAc, N-acetylmannosamine; Neu5Ac, N-acetylneuraminic acid.

Using galactosyltetrasaccharide G4 as the acceptor substrate, the donor substrate specificity study for the α2-6-sialyltransferase activity of NmSiaD_{W} was investigated using a two-step reaction. In the step 1, a CMP-sialic acid or its analog was generated in situ from a sialic acid, its analog, or its precursors in the presence of Neisseria meningitidis CMP-sialic acid synthetase (NmCSS) with or without Pasteurella multocida sialic acid aldolase (PmAldolase) and sodium pyruvate. In the step 2, galactosyltetrasaccharide G4 and NmSiaD_{W} were added. As shown in Table 2, the α2-6-sialyltransferase activity of NmSiaD_{W} was shown to tolerate different modifications at different sites on the sialic acid component in the donor substrate.

TABLE 2
Donor substrate specificity study for the α2-6-sialyltransferase activity of
NmSiaD_{W} using in-situ generated CMP-Sialic acids and analogs.

| | | Percentage conversion (%) | | |
|---|---|---|---|---|
| | | | Transferase Reaction | |
| | | | 13 μg/mL | 0.78 mg/mL |
| | | | NmSiaD_{W}, | NmSiaD_{W}, |
| | Donor Precursor | CMP-Sialic acid | 10 min | 10 h |
| 1 | Neu5Ac | Quantitative ^{a} | 60 ± 1 | Quantitative |
| 2 | Neu5Gc | 90 ± 2 ^{a} | 47 ± 3 | 88 ± 3 |
| 3 | Neu5Ac8OMe | 79 ± 1 ^{a} | 0 | 76 ± 4 |
| 4 | Neu5,9Ac | 61 ± 2 ^{a} | 0 | 0 |
| 5 | Neu4,5Ac | 53 ± 1 ^{a} | 0 | 0 |
| 6 | Kdn | Quantitative ^{a} | 0 | Quantitative |
| 7 | ManNAc6N₃ | 91 ± 10 ^{b} | 0 | 38 ± 6 |
| 8 | ManNAc4N₃ | 90 ± 3 ^{b} | 0 | 60 ± 0.3 |
| 9 | ManNAz | Quantitative ^{b} | 23 ± 1 | 86 ± 4 |
| 10 | ManNAc6NAc | 87 ± 2 ^{b} | 0 | 0 |
| 11 | Man2N₃ | 77 ± 1 ^{b} | 0 | 0 |
| 12 | 2,4-diN₃Man | 53 ± 4 ^{b} | 0 | 0 |
| 13 | 2,4,6-triN₃Man | 81 ± 1 ^{b} | 0 | 0 |

^{a} The step 1 of the reaction was carried out in the presence of NmCSS (0.75 mg/mL) for 10 h;
^{b} The step 1 of the reaction was carried out in the presence of NmCSS (0.75 mg/mL) and PmAldolase (2 mg/mL) for 10 h.
Abbreviations: Neu5Ac, N-acetylneuraminic acid; Neu5Gc, N-glycolylneuraminic acid; Neu5Ac8OMe, 8-O-methyl-N-acetylneuraminic acid; Neu5,9Ac₂, 9-O-acetyl-N-acetylneuraminic acid; Neu4,5Ac₂, 4-O-acetyl-N-acetylneuraminic acid; Kdn, 2-keto-3-deoxynonulosonic acid; ManNAc6N₃, 6-azido-6-deoxy-N-acetylmannosamine; ManNAc4N₃, 4-azido-4-deoxy-N-acetylmannosamine; ManNAz, N-azidoacetylmannosamine; ManNAc6NAc, 6-N-acetyl-6-deoxy-N-acetylmannosamine; Man2N₃, 2-azido-2-deoxy-mannose; 2,4-diN₃Man, 2,4-diazido-2,4-dideoxy-mannose; 2,4,6-triN₃Man, 2,4,6-triazido-2,4,6-trideoxy-mannose.

Using UDP-Gal as the donor substrate, the acceptor substrate specificity for the α1-4-galactosyltransferase activity of NmSiaD_{W} was studied using eleven sialosides. As shown in Table 3, Neu5AcαOMe as well as α2-3- and α2-6-linked sialosides containing Neu5Ac or its derivatives were suitable acceptor substrates. Quite interestingly, the α1-4-galactosyltransferase activity of NmSiaD_{W} could also tolerate Neu5Acα2-3Galβ1-3GalNAcβProN₃ (Entry 9 in Table 3) for the synthesis of the tetrasaccharide repeating unit in E. coli serotype K9 capsular polysaccharide.

TABLE 3
Acceptor substrate specificity study of the αl-4-galactosyltransferase
activity of NmSiaD_{W} using sialosides as potential acceptors.

| | Sialoside | Product |
|---|---|---|
| 1 | Neu5Acα2-6GalβpNP | √ |
| 2 | Neu5Acα2-6GalβProNH₂ | √ |
| 4 | Neu5Acα2-6Galβ1-4βProN₃ | √ |
| 5 | Neu5AcαOMe | √ |
| 5 | Neu5Ac9NAcα2-6GalβpNP | √ |
| 6 | Neu5Ac9NAcα2-6GalβProN₃ | √ |
| 7 | Neu5Ac7N₃α2-6Galα1-4Neu5AcαProNHCbz | √ |
| 8 | Neu5Ac9N₃α2-6Galα1-4Neu5AcαProNHCbz | √ |
| 9 | Neu5Acα2-3Galβ1-3GalNAcβProN₃ | √ |
| 10 | Leg5,7diN₃α2-3Galβ1-3GalNAcβProCl | √ |
| 11 | Leg5,7Ac₂α2-3Galβ1-3GalNAcβProN₃ | Not Detected |

Using CMP-Neu5Ac as the donor substrate, the acceptor substrate specificity for the α2-6-sialyltransferase activity of NmSiaD_{W} was studied using six galactosides. As shown in Table 4, β-linked galactosylmonosaccharide (Entry 5 in Table 4) and β1-4-linked galactosyldisaccharides (Entries 2-4 in Table 4) was shown to be suitable acceptors. While α1-4-linked galactosyltrisaccharide (Entry 1 in Table 4) was a suitable acceptor, a-linked monosaccharide (Entry 6 in Table 4) was not tolerated.

TABLE 4
Acceptor substrate specificity study for the α2-6-sialyltransferase
activity of NmSiaD_{W} using galactosides as potential acceptors.

| | Galactoside | Product |
|---|---|---|
| 1 | Galα1-4Neu5Acα2-6GalβpNP | √ |
| 2 | Galβ1-4Glc | √ |
| 3 | Galβ1-4GlcβMU | √ |
| 4 | Galβ1-4Glcβ2AA | √ |
| 5 | GalβProN₃ | √ |
| 6 | GalαOMe | Not Detected |

### Example 5. Kinetics Studies

A. Experimental Procedure

Enzyme kinetics by varying acceptor concentrations. For galactosyltransferase acceptors, reactions were performed in duplicate at 30° C. for 10 minutes in the presence of MES buffer (100 mM, pH 6.5), MgCl₂ (10 mM) and 2 mM UDP-Gal with a total volume of 20 μL, and varying concentrations (0.05, 0.1, 0.2, 0.3, 0.5, 0.7, 1.0, 2.0, 5.0 and 10.0 mM) of the acceptor substrate. The concentration of NmSiaD_{W} varied from 0.011 to 4.049 μM when different acceptors were used. Reactions were quenched by adding 20 Å of pre-chilled ethanol followed by incubation at −20° C. for 30 min. The apparent kinetic parameters were obtained by fitting the experimental data (the average values of duplicate assay results) into the Michaelis-Menten equation using Grafit 5.0.

For sialyltransferase acceptors, reactions were performed in duplicate at 30° C. for 10 minutes in the presence of 100 mM Tris-HCl, pH 8.0, 10 mM MgCl₂ and 10 mM CMP-Neu5Ac with a total volume of 20 μL, and varying concentrations (0.1, 0.2, 0.3, 0.5, 0.7, 1.0, 1.5, 2.0, 3.0, 5.0 and 10.0 mM) of the acceptor substrate. The concentration of NmSiaD_{W} varied from 0.011 to 0.018 μM according to different acceptors. Reactions were quenched by adding 20 μL of pre-chilled ethanol followed by incubation at −20° C. for 30 min. Products were assayed using an Agilent 1290 Infinity II LC System with a PDA detector (monitored at 215 nm) and an Eclipse Plus C18 column (Rapid Resolution HID, 1.8 μm, 2.1×50 mm, 959757-902) or an AdvanceBio Glycan Map column (1.8 μm, 2.1×150 mm, 859700-913) (see Table 5 below for detailed elution conditions) at 30° C. The apparent kinetic parameters were obtained by fitting the experimental data (the average values of duplicate assay results) into the Michaelis-Menten equation using Grafit 5.0.

TABLE 5
Elution conditions for NmSiaD_{W} kinetics studies with different acceptors (S1-G10).

| | [E] | | | | |
|---|---|---|---|---|---|
| Acceptor | (μM) | Column | Solvent A | Solvent B | B % |
| S1 | 4.049 | Eclipse Plus | 0.1% TFA in H₂O | Acetonitrile | 11 |
| | | C18 | | | |
| G2 | 0.014 | AdvanceBio | 35 mM NaCl, | Acetonitrile | 88 |
| | | Glycan | 0.1% TFA in H₂O | | |
| S3 | 0.048 | AdvanceBio | 35 mM NaCl, | Acetonitrile | 88-84 |
| | | Glycan | 0.1% TFA in H₂O | | over 4 min |
| G4 | 0.011 | Eclipse Plus | 10 mM tetrabutylammonium, 50 | Acetonitrile | 27-34 |
| | | C18 | mM ammonium formate, pH 4.5 | | over 4 min |
| S5 | 0.018 | AdvanceBio | 35 mM NaCl, | Acetonitrile | 75 |
| | | Glycan | 0.1% TFA in H₂O | | |
| G6 | 0.014 | AdvanceBio | 35 mM NaCl, | Acetonitrile | 75 |
| | | Glycan | 0.1% TFA in H₂O | | |
| S7 | 0.018 | AdvanceBio | 35 mM NaCl, | Acetonitrile | 77-72 |
| | | Glycan | 0.1% TFA in H₂O | | over 5 min |
| G8 | 0.018 | AdvanceBio | 35 mM NaCl, | Acetonitrile | 72 |
| | | Glycan | 0.1% TFA in H₂O | | |
| S9 | 0.011 | AdvanceBio | 35 mM NaCl, | Acetonitrile | 71 |
| | | Glycan | 0.1% TFA in H₂O | | |
| G10 | 0.014 | AdvanceBio | 35 mM NaCl, | Acetonitrile | 70 |
| | | Glycan | 0.1% TFA in H₂O | | |

Enzyme kinetics by varying donor concentrations. For varying UDP-Gal concentrations, reactions were performed in duplicate at 30° C. for 10 minutes in the presence of MES buffer (100 mM, pH 6.5), MgCl₂ (10 mM), S3 or S9 (2 mM), UDP-Gal (0.1, 0.2, 0.5, 1.0, 2.0, 5.0 and 10.0 mM), and NmSiaD_{W} (0.025 μM for S3, 0.031 μM for S9) in a total volume of 20 μL. For varying CMP-Neu5Ac concentrations, reactions were performed in duplicate at 30° C. for 10 minutes in the presence of Tris-HCl buffer (100 mM, pH 8.0), MgCl₂ (10 mM), G2 or G10 (1 mM), CMP-Neu5Ac (0.2, 0.5, 1.0, 2.0, 5.0 and 10.0 mM), and NmSiaD_{W} (0.025 μM for G2, 0.062 μM for G10) in a total volume of 20 μL. Data analyses were carried out as described above.

B. Results

The synthesized Cbz-tagged monosaccharide and oligosaccharides of varied lengths (S1-G10) were used as acceptors for kinetics studies of NmSiaD_{W}. Two distinctive kinetics behaviors were observed for two glycosyltransferase activities. For the galactosyltransferase activity using sialosides S1-S9, the catalytic efficiency (k_{cat}/K_{M}) was significantly improved as the length of the acceptor substrate increased. The improvement is mainly resulted from a higher binding affinity of the acceptor substrate. The overall catalytic efficiency increased more than 2100-fold from monosaccharide S1 to nonasaccharide S9 (Table 6A).

The K_{M} of nonasaccharide S9 kept decreasing to lower than 0.05 mM. Since the lowest concentration in assay was 0.05 mM due to the absorptivity of Cbz tag, the calculated K_{M} was not reliable. Substrate inhibition was found when the concentration of nonasaccharide was higher than 2 mM. Substrate inhibition may result from the extremely low K_{M} of the nonasaccharide.

Acceptor substrate inhibition was observed when the concentration of S9 was higher than 2 mM. This can be explained by the low K_{M} value of S9 which may compete with the donor binding to the enzyme, inhibiting an effective catalytic process by glycosyltransferases which follows an ordered sequential Bi-Bi mechanism where the enzyme binds the sugar nucleotide before the acceptor. On the other hand, the k_{cat} (5.1-8.8 s⁻¹) did not change significantly as the length of the sialoside acceptor varied. A similar preference for longer acceptor substrates was demonstrated previously for a GT4 family glucosyltransferase using lipid acceptors.

For the sialyltransferase activity using G2-G10 as the acceptors, it was found that the galactoside acceptors showed substrate inhibition activity from the disaccharide with 2 mM CMP-Neu5Ac as donor. Substrate inhibition may result from the ordered binding mode of glycosyltransferase. To overcome substrate inhibition for fitting in the Michaelis-Menten equation, the concentration of CMP-Neu5Ac was increased to 10 mM. The enzyme activity was slightly recovered around 1-2 mM acceptor (FIG. 4).

When using galactosides G2-G10 as acceptors, the catalytic efficiency (k_{cat}/K_{M}) of NmSiaD_{W} α2-6-sialyltransferase activity was shown to be in a narrow range of 46-97 s⁻¹ mM⁻¹ without significant change as the length of the acceptor substrate was varied (Table 6B). The k_{cat} was in a range of 7.03-23.1 s⁻¹ and the K_{M} fell in the range of 0.13-0.24 mM. When G4 was used as the acceptor, NmSiaD_{W} α2-6-sialyltransferase activity had the highest catalytic efficiency (97 s⁻¹ mM⁻¹) compared to the other four acceptors (k_{cat} K_{M}=46-55 s⁻¹ mM⁻¹) mainly due to a relatively higher k_{cat} (23.1+1.1 s⁻¹) than those of G2, G6, G8, and G10 (7.03-11.9 s⁻¹).

TABLE 6
Apparent kinetics data for NmSiaD_{W} galactosyltransferase
activity (A) and sialyltransferase activity (B)

| | | | |
|---|---|---|---|
| Acceptor | k_{cat} (s⁻¹) | K_{M} (mM) | k_{cat}/K_{M} (s⁻¹ mM⁻¹) |

(A)

| | | | |
|---|---|---|---|
| S1 | / | >>10.0 | 4.7 × 10⁻² |
| S3 | 8.8 ± 0.4 | 0.89 ± 0.10 | 10 |
| S5 | 5.3 ± 0.2 | 0.10 ± 0.02 | 51 |
| S7 | 5.5 ± 0.2 | 0.07 ± 0.01 | 83 |
| S9 | 5.1 ± 0.3 | <0.05 | >1.0 × 10² |

(B)

| | | | |
|---|---|---|---|
| G2 | 9.76 ± 0.55 | 0.18 ± 0.04 | 55 |
| G4 | 23.1 ± 1.1 | 0.24 ± 0.04 | 97 |
| G6 | 11.9 ± 0.5 | 0.23 ± 0.04 | 53 |
| G8 | 10.3 ± 0.5 | 0.25 ± 0.04 | 46 |
| G10 | 7.03 ± 0.39 | 0.13 ± 0.03 | 55 |

NmSiaD_{W} kinetics studies where the concentrations of the donors were also conducted, using a fixed concentration of a representative short or long acceptor substrate. S3 or S9 was used as the acceptor substrate for varying the concentration of UDP-Gal and G2 or G10 was used as the acceptor substrate for varying the concentration of CMP-Neu5Ac. As shown below, the kinetics parameters for the α1-4-galactosyltransferase (Table 7) and the α2-6-sialyltransferase (Table 8) activities of NmSiaD_{W} did not change significantly when different sizes of acceptors were used. Table 7 shows apparent kinetics data for NmSiaD_{W}α1-4-galactosyltransferase activity using a fixed concentration of acceptor (S3 or S9). Table 8 shows apparent kinetics data for NmSiaD_{W} α2-6-sialyltransferase activity using a fixed concentration of acceptor (G2 or G10). The averages of nonlinear regression standard errors from technical duplicates are shown.

TABLE 7

| Acceptor | k_{cat} (s⁻¹) | K_{M} (mM) | k_{cat}/K_{M} (s⁻¹ mM⁻¹) |
|---|---|---|---|
| S3 | 6.3 ± 0.2 | 0.12 ± 0.02 | 53 |
| S9 | 9.0 ± 0.2 | 0.15 ± 0.02 | 60 |

TABLE 8

| Acceptor | k_{cat} (s⁻¹) | K_{M} (mM) | k_{cat}/K_{M} (s⁻¹ mM⁻¹) |
|---|---|---|---|
| G2 | 7.1 ± 0.2 | 0.27 ± 0.03 | 26 |
| G10 | 6.1 ± 0.2 | 0.38 ± 0.05 | 16 |

### Example 6. Polymerization Study

A. Experimental Method

For studies using acceptor substrates of varied lengths, reactions were performed in duplicate in a total volume of 50 μL at 30° C. in Tris-HCl buffer (100 mM, pH 8.5) containing MgCl₂ (10 mM), UDP-Gal (50 mM), CMP-Neu5Ac (50 mM), an acceptor substrate (5 mM, selected from S1-G10) and NmSiaD_{W} (50 μg).

For donor ratio profile studies using G2 or S3 as the acceptor substrate, reactions were performed in duplicate in a total volume of 50 μL at 30° C. in Tris-HCl buffer (100 mM, pH 8.5) containing MgCl₂ (10 mM), both UDP-Gal and CMP-Neu5Ac (5, 10, 25, 50, 100 and 250 mM), an acceptor substrate G2 or S3 (5 mM), and NmSiaD_{W} (50 μg).

For one-pot multienzyme (OPME) polymerization studies using G2 or S3 as the acceptor substrate, reactions were performed in two steps. A donor synthesis reaction was carried out in a total volume of 150 μL at 30° C. for 10 hours in Tris-HCl buffer (144 mM, pH 8.5) containing MgCl₂ (14.4 mM), CTP (72 mM), Neu5Ac (72 mM), UTP (72 mM), ATP (72 mM), Gal (72 mM), SpGalK (100 μg), BLUSP (50 μg), PmPpA (100 μg) and NmCSS (80 μg). Then polymerization reactions were performed in duplicate in a total volume of 50 μL each at 30° C. containing a reaction mixture of the donor synthesis (35 μL), G2 or S3 (5 mM), and NmSiaD_{W} (50 μg). Samples were taken and quenched at 1 h and 20 h, respectively, by transferring 20 μL of reaction mixture into an equal volume of pre-chilled ethanol followed by incubation at −20° C. for 30 min.

Reaction mixtures were analyzed using UHPLC (monitored at 215 nm) with an AdvanceBio Glycan Map column (a HILIC column from Agilent, 1.8 μm, 2.1×150 mm, 859700-913) at 30° C. Solvent A (35 mM NaCl, 0.1% TFA in H₂O) and solvent B (acetonitrile) were used to establish an elution gradient, starting with 90% B at 1.300 mL/min and reaching to 40% B at 0.675 mL/min over 50 minutes. Relative yields were calculated from peak area integration and used to obtain number average molecular weight, weight average molecular weight, and polydispersity index.

Reaction mixtures were analyzed at 30° C. using a Shimadzu LCMS-2020 system (monitored at 215 nm) with a XBridge BEH Amide Column (a HILIC column from Waters, 130 Å, 5 μm, 4.6×250 mm). Solvent A (0.1% formic acid in H₂O) and solvent B (acetonitrile) were used to establish an elution gradient, starting with 72.5% B at 1.300 mL/min and reaching to 12.5% B at 0.800 mL/min over 120 minutes.

B. Results

Despite the product profiles for several Nm glycosyltransferases have been well studied (see, e.g., Keys 2014; Fiebig 2018), study of polymerization for heteropolymers of Nm capsular polysaccharides is lacking. There are multiple concerns to be considered, including different reaction conditions, donor ratio, detection method, etc. Synthetic conditions with UDP-Gal and CMP-Neu5Ac can be applied to determine the product profile of NmSiaD_{W}.

The availability of chromophore-tagged NmW CPS mono- and oligosaccharides with defined sizes and structures (S1-G10) allowed us to address several questions. Does the length of NmSiaD_{W} oligosaccharide acceptor affect the maximal product sizes when both donors are provided? Does the identity of the monosaccharide at the reducing end of the oligosaccharide acceptor affect the maximal product sizes? What is the effect of the donor versus acceptor ratio on the product size distribution?

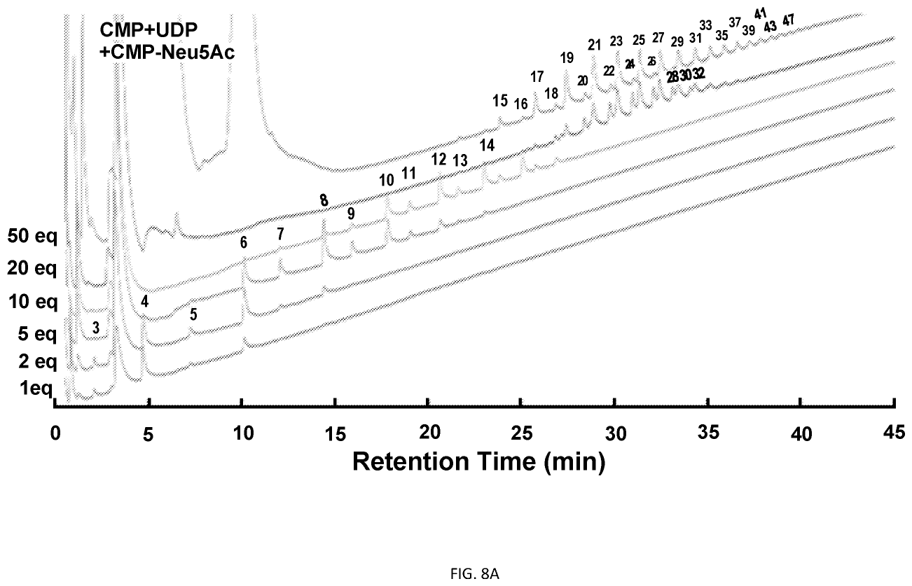

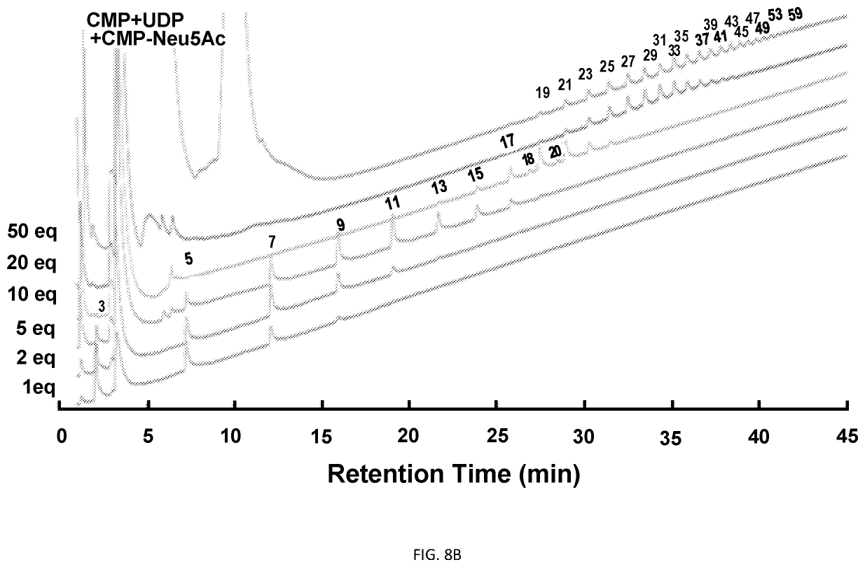

To answer the first two questions, NmSiaD_{W}-catalyzed polymerization reactions were carried out with an acceptor (5 mM) selected from S1-G10 and 10 equivalents of both UDP-Gal and CMP-Neu5Ac donors. Reaction mixtures were analyzed using an UHPLC system with an AdvanceBio Glycan Mapping column (a HILIC column) using NaCl and acetonitrile gradients. Except for the reactions using S1 as the acceptor which were slow, no significant difference on the maximal product sizes was observed when oligosaccharide acceptors (G2-G10) of different sizes were used. Nevertheless, compared to reactions with a shorter acceptor (G2-S7), a narrower product size distribution was seen for reactions with a longer oligosaccharide acceptor (G8, S9, or G10). Therefore, using longer oligosaccharide acceptors (G8-G10) could be advantages for the production of monodisperse NmW capsular polysaccharides. The identity of the reducing-end monosaccharide did not seem to affect the maximal product sizes either. It was interesting to observe (see, e.g., FIG. 8) that sialosides seemed to be the preferred products when a sialoside was used as the starting acceptor substrate. In comparison, a galactoside starting acceptor led to the formation of both galactoside and sialoside products. The underlying reason could be related to the difference in the relative availability of two donor substrates in the reaction mixtures.

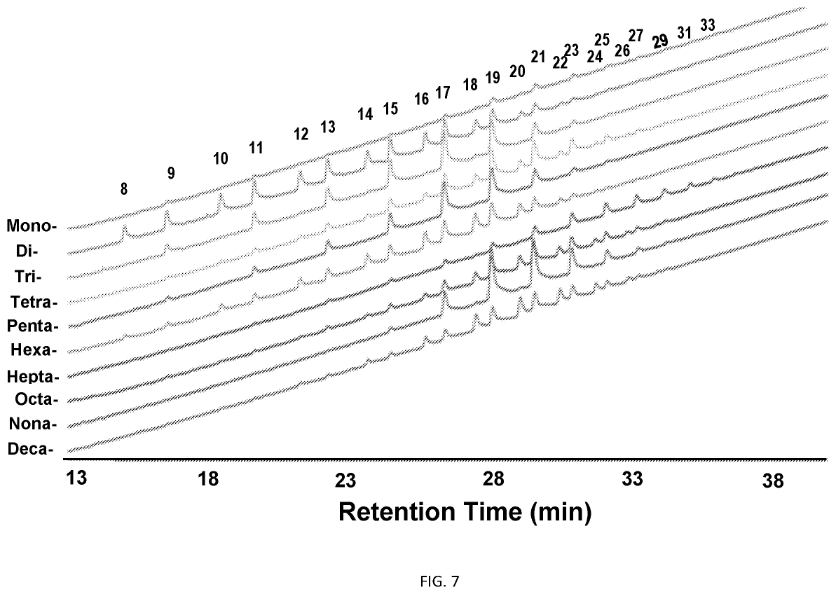

With 10 equivalents of both UDP-Gal and CMP-Neu5Ac in 20-hour reactions, the longest product tended to be degree of polymerization (DP) 33 (FIG. 7). The most abundant products are between DP17 to DP23. Although the product profile is slightly influenced by the starting acceptor, size distribution is similar among the 10 oligosaccharide acceptors tested. One exception was observed for monosaccharide due to the difficulty to produce disaccharide as the first step, the same as the problem found in one-pot four-enzyme galactosylation. However, differences occurred between sialoside and galactoside acceptors. Sialoside acceptors (mono-, tri-, penta-, hepta- and nona-saccharide) always resulted in sialoside products with an odd DP value. But the galactoside acceptors (di-, tetra-, hexa-, octa-, and deca-saccharide) ended with both galactoside and sialoside products with similar levels. The mechanism behind the product distribution may depend on different kinetic behaviors.

The effect of the donor verses acceptor ratio on the product size distribution was investigated using a series of ratios varying from 1 to 50 with either G2 or S3 as the acceptor. As shown in FIG. 8, the sizes of the products increased with the increase of the donor versus acceptor ratio independent of whether a galactoside G2 or a sialoside S3 was used as the acceptor. Polymers with DP59 or higher were observed. In comparison, NmSiaD_{W} was reported to form products for up to DP19 using Neu5Acα2-6Galα1-4Neu5AcaMU as the acceptor and 4 equivalents of both donors. The strategy of using a high donor versus acceptor ratio was also applied previously for synthesizing monodisperse polysaccharides such as hyaluronan (up to 8 MDa) using Pasteurella multocida hyaluronan synthase (PmHAS) and heparosan (800 kDa) using Pasteurella multocida heparosan synthase 1 (PmHS1).

Assuming oligosaccharides S3-G8 were not part of the products in the 20-h reactions using 50 equivalents of donors (FIG. 8), more detailed analyses showed that when G2 was used as the acceptor substrate, the average molecular weights (Mₙ or M_{w}) of NmSiaD_{W} products increased from 1.0 kDa to 6.1-6.6 kDa when the donor versus acceptor ratio changed from 1 to 50 (Table 9) and the product average molecular weights increased from 1.4 kDa to 7.5-8.6 kDa when S3 was used as the acceptor substrate (Table 10). NmSiaD_{W} catalyzed the formation of low molecular weight polysaccharides with a narrow size distribution (polydispersity index: M_{w}/Mₙ=1.03-1.14) under the experimental conditions used. Table 9 shows the average molecular masses and polydispersity of product profiles of 20-hour reactions using different ratios (1-50 equivalents) of donors versus G2 (5 mM) in FIG. 8A. Table 10 shows the average molecular masses and polydispersity of product profiles of 20-hour reactions using different ratios (1-50 equivalents) of donors versus S3 (5 mM) in FIG. 8B.

TABLE 9

| Donor | | | | | | |
|---|---|---|---|---|---|---|
| Equivalents | 1 | 2 | 5 | 10 | 20 | 50 |
| Mₙ | 951 | 1310 | 2135 | 3072 | 5325 | 6050 |
| (g/mol) | | | | | | |
| M_{w} | 1051 | 1416 | 2237 | 3215 | 5487 | 6609 |
| (g/mol) | | | | | | |
| PDI | 1.11 | 1.08 | 1.05 | 1.05 | 1.03 | 1.09 |

Mₙ: number average molecular mass;
M_{w}: mass average molecular mass;
PDI: polydispersity index, PDI = M_{w}/Mₙ.

TABLE 10

| Donor | | | | | | |
|---|---|---|---|---|---|---|
| Equivalents | 1 | 2 | 5 | 10 | 20 | 50 |
| Mₙ (g/mol) | 1333 | 1704 | 2567 | 4313 | 7051 | 7526 |
| M_{w} (g/mol) | 1453 | 1888 | 2870 | 4436 | 7272 | 8554 |
| PDI | 1.09 | 1.11 | 1.12 | 1.03 | 1.03 | 1.14 |

Mₙ: number average molecular mass;
M_{w}: mass average molecular mass;
PDI: polydispersity index, PDI = M_{w}/Mₙ.

The application of in situ generation of sugar nucleotide donors (UDP-Gal and CMP-Neu5Ac) by OPME galactosylation and sialylation systems in polymerization reaction was investigated using G2 or S3 as the acceptor substrate and compared to the reactions using 10 equivalents of donor substrates. The OPME polymerization reactions were carried out in two steps where the sugar nucleotides were formed at 30° C. for 10 hours in Tris-HCl buffer from ATP, UTP, Gal, CTP, Neu5Ac at pH 8.5 in the presence of SpGalK, BLUSP, PmPpA, and NmCSS. The reaction mixture was then added with G2 or S3, and NmSiaD_{W} for polymerization reactions. Polymerization reactions with OPME systems were slower but reached similar levels as those using sugar nucleotides as starting materials in a 20-h reaction time (data not shown).

Chemoenzymatic reaction provides a green and efficient method to synthesize pathogenic capsular polysaccharide in preparative-scale. Previously, a total synthesis method was employed to achieve 35-50% yield in three steps. See, Wang 2013. With the high-efficiency one-pot multi-enzyme system provided herein, the yield can be higher than 80% after a single-step reaction. The reaction is undertaken on 200-450 mg scale, with the ability to enlarge to gram-scale synthesis. With the chemoenzymatic method according to the present disclosure, both galactoside and sialoside products can be obtained with an efficient manner, while only the sialoside products were obtained based on the previous report. Immunology studies can directly benefit from a full library of bacterial capsular oligosaccharides and can further guide a rational vaccine development based on the length of the oligosaccharide.

As described above, recombinant NmSiaD_{W} was expressed in a high expression level, characterized in detail, and applied in synthesis. A library of NmW capsular polysaccharides were synthesized using one-pot multienzyme (OPME) chemoenzymatic glycosylation systems with high efficiency (83-96%). Kinetics studies indicated that galactosides inhibited the sialyltransferase activity of the enzyme. The catalytic efficiency of the galactosyltransferase activity increased with the increased length of the sialoside acceptors. More than 2300-fold improvement was observed when the acceptor length increased from monosaccharide to nonasaccharide. Substrate inhibition was also found in nonasaccharide. NmSiaD_{W} was shown to be a promiscuous enzyme by a preliminary screening using libraries of potential donors and acceptors containing different sugars.

### IV. EXEMPLARY EMBODIMENTS

Exemplary embodiments provided in accordance with the presently disclosed subject matter include, but are not limited to, the claims and the following embodiments:
1. A method for preparing a bacterial capsular saccharide product, the method comprising:
forming a reaction mixture containing one or more bacterial capsular polysaccharide synthases, a sugar acceptor, and one or more sugar donors; and maintaining the reaction mixture under conditions sufficient to form the bacterial capsular saccharide product;
wherein the degree of polymerization of the bacterial capsular saccharide product ranges from 2 to about 200, and wherein the polydispersity index M_{w}/Mₙ of the bacterial capsular saccharide product ranges from 1 to about 1.5.
2. The method of embodiment 1, wherein the bacterial capsular saccharide product is a heteropolymer comprising disaccharide repeating units.
3. The method of embodiment 1 or embodiment 2, wherein forming the bacterial capsular saccharide product comprises glycosylating the sugar acceptor with monosaccharide residues of a first variety and monosaccharide residue of a second variety in alternating steps.
4. The method of embodiment 1 or embodiment 2, wherein forming the bacterial capsular saccharide product comprises glycosylating the sugar acceptor with alternating monosaccharide residues of a first variety and monosaccharide residues of a second variety in a single polymerization step.
5. The method of any one of embodiments 1-4, the degree of polymerization of the bacterial capsular saccharide product ranges from 20 to about 200.
6. The method of any one of embodiments 1-5, wherein the degree of polymerization of the bacterial capsulate saccharide product is greater than 50.
7. The method of any one of embodiments 1-6, wherein the polydispersity index M_{w}/Mₙ of the bacterial capsular saccharide product ranges from 1.01 to about 1.15
8. The method of any one of embodiments 1-7, wherein each bacterial capsular polysaccharide synthase is independently selected from N. meningitidis SiaD_{w}(NmSiaD_{W}), N. meningitidis SiaD_{y} (NmSiaD_{Y}), a P. multocida heparosan synthase (PmHS1 and PmHS2), P. multocida hyaluronan synthase (PmHAS), S. pyogenes hyaluronan synthase (SpHAS), P. multocida chondroitin synthase (PmCS), E. coli K5 KfiA and KfiC, S. pneumoniae Type 3 capsular polysaccharide synthase (SpCps3S), and S. pneumoniae Type 37 capsular polysaccharide synthase (SpCps37Tts).
9. The method of embodiment 8, wherein the reaction mixture comprises one bacterial capsular polysaccharide synthase, and wherein the bacterial capsular polysaccharide synthase is NmSiaD_{W}.
10. The method of any one of embodiments 1-9, wherein the bacterial capsular saccharide product comprises galactose-sialic acid disaccharide repeating units.
11. The method of embodiment 10, wherein the galactose-sialic acid disaccharide repeating units are (-6Galα1-4Neu5Acα2).
12. The method of embodiment 10 or embodiment 11, wherein the reaction mixture comprises a galactose donor, a sialic acid donor, or a combination thereof.
13. The method of embodiment 12, wherein the galactose donor is UDP-Gal.
14. The method of embodiment 12 or embodiment 13, wherein the sialic acid donor is CMP-Neu5Ac.
15. The method of any one of embodiments 10-14, wherein forming the bacterial capsular saccharide product comprises glycosylating the sugar acceptor with galactose residues and sialic acid residues in alternating steps.
16. The method of any one of embodiments 10-14, wherein forming the bacterial capsular saccharide product comprises glycosylating the sugar acceptor with alternating galactose residues and sialic acid residues in a single polymerization step.
17. The method of embodiment 16, wherein the reaction mixture comprises UDP-Gal and CMP-Neu5Ac, and wherein the ratio (UDP-Gal+CMP-Neu5Ac):(sugar acceptor) ranges from about 1:1 to about 250:1.
18. The method of embodiment 17, wherein the ratio is about 100:1.
19. The method of any one of embodiments 1-18, wherein the sugar acceptor comprises a sialic acid residue at its non-reducing end.
20. The method of any one of embodiments 1-18, wherein the sugar acceptor comprises a galactose residue at its non-reducing end.
21. The method of any one of embodiments 1-20, wherein the sugar acceptor comprises an oligosaccharide moiety Galα1-4Neu5Acα2(-6Galα1-4Neu5Acα2)ₙ- or an oligosaccharide moiety Neu5Acα2(-6Galα1-4Neu5Acα2)ₘ-, wherein subscript n is 1, 2, 3, or 4 and subscript m is 1, 2, 3, 4, or 5.
22. The method of any one of embodiments 1-21, wherein the acceptor comprises a purification handle.
23. The method of any one of embodiments 1-22, wherein the reaction mixture further comprises a CMP-sialic acid synthetase, a nucleotide sugar pyrophosphorylase, a pyrophosphatase, a kinase, or a combination thereof.
24. The method of embodiment 23, wherein the CMP-sialic acid synthetase is NmCSS, wherein the nucleotide sugar pyrophosphorylase is BLUSP, wherein the pyrophosphatase is PmPpA, and wherein the kinase is SpGalK.
25. The method of any one of embodiments 1-24, wherein the pH of the reaction mixture ranges from about 6 to about 9.
26. The method of any one of embodiments 1-24, which is conducted in vitro.
27. A bacterial capsular saccharide product prepared according to the method of any one of embodiments 1-26.
28. A vaccine composition comprising a bacterial capsular saccharide product prepared according to the method of any one of embodiments 1-26 coupled to a carrier material.

### V. REFERENCES

Aguilera, J.-F. et al. Emerging infectious diseases 2002, 8, 761-767.
Batista, R. S. et al. Asian Pac J Trop Med 2017, 10 (11), 1019-1029.
Bergfeld, A. K. et al. J Biol Chem 2009, 284 (1), 6-16.
Boix, E. et al. J Biol Chem 2002, 277 (31), 28310-8.
Brouwer, M. C. et al. Curr Opin Infect Dis 2018, 31 (1), 78-84.
Chen, M. et al. Carbohydr Res 2011, 346 (15), 2421-5.
Chen, X. et al. ACS Chem Biol 2010, 5 (2), 163-76.
Claus, H. et al. Mol Microbiol 2009, 71 (4), 960-71.
Claus, H. et al. Mol Gen Genet 1997, 257, 28-34.
Fiebig, T. et al. J Biol Chem 2018, 293 (3), 953-962.
Gasparini, R. et al. Human Vaccines 2014, 7 (2), 170-182.
Gudlavalleti, S. K. et al. J Pharm Biomed Anal 2015, 107, 432-6.
Harrison, L. H. et al. Nat Rev Drug Discov 2010, 9 (6), 429-30.
Keys, T. G. et al. Nat Chem Biol 2014, 10 (6), 437-42.
Kopycki, J. et al. Biochem J 2013, 450 (1), 37-46.
Lairson, L. L. et al. Annu Rev Biochem 2008, 77, 521-55.
Lau, K.; Yu, H.; Chen, X.; et al. Chem Commun (Camb) 2010, 46 (33), 6066-8.
Li, W.; Yu, H.; Chen, X.; et al. Carbohydr Res 2017, 451, 51-58.
Livermore, D. M. Int J Antimicrob Agents 2012, 39 (4), 283-94.
Mishra, R. P. et al. Curr Opin Microbiol 2012, 15 (5), 596-602.
Muthana, M. M.; Yu, H.; Chen, X.; et al. Chem Commun (Camb) 2012, 48 (21), 2728-30.
Romanow, A. et al. J Biol Chem 2013, 288 (17), 11718-30.
Romanow, A. et al. J Biol Chem 2014, 289 (49), 33945-57.
Rouphael, N. G. et al. Methods Mol Biol 2012, 799, 1-20.
Sardzik, R. et al. Chem Commun (Camb) 2011, 47 (19), 5425-7.
Sharyan, A. et al. BMC Res Notes 2018, 11 (1), 482.
Wang,. et al. Angew Chem Int Ed Engl 2013, 52 (35), 9157-61.
Yoshino, M. et al. Springerplus 2015, 4, 292.
Yu, H.; Chen, X.; et al. Bioorg Med Chem 2004, 12 (24), 6427-35.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

INFORMAL SEQUENCE LISTING
(NmSiaDw)
SEQ ID NO: 1
MAVIIFVNGIRAVNGLVKSSINTANAFAEEGLDVH
LINFVGNITGAEHLYPPFHLHPNVKTSSIIDLFND
IPENVSCRNTPFYSIHQQFFKAEYSAHYKHVLMKI
ESLLSAEDSIIFTHPLQLEMYRLANNDIKSKAKLI
VQIHGNYMEEIHNYEILARNIDYVDYLQTVSDEML
EEMHSHFKIKKDKLVFIPNITYPISLEKKEADFFI
KDNEDIDNAQKFKRISIVGSIQPRKNQLDAIKIIN
KIKNENYILQIYGKSINKDYFELIKKYIKDNKLQN
RILFKGESSEQEIYENTDILIMTSESEGFPYIFME
GMVYDIPIVVYDFKYGANDYSNYNENGCVFKTGDI
SGMAKKIIELLNNPEKYKELVQYNHNRFLKEYAKD
VVMAKYFTILPRSFNNVSLSSAFSRKELDEFQNIT
FSIEDSNDLAHIWNFELTNPAQNMNFFALVGKRKF
PMDAHIQGTQCTIKIAHKKTGNLLSLLLKKRNQLN
LSRGYTLIAEDNSYEKYIGAISNKGNFEIIANKKS
SLVTINKSTLELHEIPHELHQNKLLIALPNMQTPL
KITDDNLIPIQASIKLEKIGNTYYPCFLPSGIFNN
ICLDYGEESKIINFSKYSYKYIYDSIRHIEQHTDI
SDIIVCNVYSWELIRASVIESLMEFTGKWEKHFQT
SPKIDYRFDHEGKRSMDDVFSEETFIMEFPRKNGI
DKKTAAFQNIPNSIVMEYPQTNGYSMRSHSLKSNV
VAAKHFLEKLNKIKVDIKFKKHDLANIKKMNRIIY
EHLGININIEAFLKPRLEKFKREEKYFHDFFKRNN
FKEVIFPSTYWNPGIICAAHKQGIKVSDIQYAAIT
PYHPAYFKSPKSHYVADKLFLWSEYWNHELLPNPT
REIGSGAAYWYALDDVRFSEKLNYDYIFLSQSRIS
SRLLSFAIEFALKNPQLQLLFSKHPDENIDLKNRI
IPDNLIISTESSIQGINESRVAVGVYSTSLFEALA
CGKQTFVVKYPGYEIMSNEIDSGLFFAVETPEEML
EKTSPNWVAVADIENQFFGQEK
(NmSiaDw-Hise)
SEQ ID NO: 2
MAVIIFVNGIRAVNGLVKSSINTANAFAEEGLDVH
LINFVGNITGAEHLYPPFHLHPNVKTSSIIDLFND
IPENVSCRNTPFYSIHQQFFKAEYSAHYKHVLMKI
ESLLSAEDSIIFTHPLQLEMYRLANNDIKSKAKLI
VQIHGNYMEEIHNYEILARNIDYVDYLQTVSDEML
EEMHSHFKIKKDKLVFIPNITYPISLEKKEADFFI
KDNEDIDNAQKFKRISIVGSIQPRKNQLDAIKIIN
KIKNENYILQIYGKSINKDYFELIKKYIKDNKLQN
RILFKGESSEQEIYENTDILIMTSESEGFPYIFME
GMVYDIPIVVYDFKYGANDYSNYNENGCVFKTGDI
SGMAKKIIELLNNPEKYKELVQYNHNRFLKEYAKD
VVMAKYFTILPRSFNNVSLSSAFSRKELDEFQNIT
FSIEDSNDLAHIWNFELTNPAQNMNFFALVGKRKF
PMDAHIQGTQCTIKIAHKKTGNLLSLLLKKRNQLN
LSRGYTLIAEDNSYEKYIGAISNKGNFEIIANKKS
SLVTINKSTLELHEIPHELHQNKLLIALPNMQTPL
KITDDNLIPIQASIKLEKIGNTYYPCFLPSGIFNN
ICLDYGEESKIINFSKYSYKYIYDSIRHIEQHTDI
SDIIVCNVYSWELIRASVIESLMEFTGKWEKHFQT
SPKIDYRFDHEGKRSMDDVFSEETFIMEFPRKNGI
DKKTAAFQNIPNSIVMEYPQTNGYSMRSHSLKSNV
VAAKHFLEKLNKIKVDIKFKKHDLANIKKMNRIIY
EHLGININIEAFLKPRLEKFKREEKYFHDFFKRNN
FKEVIFPSTYWNPGIICAAHKQGIKVSDIQYAAIT
PYHPAYFKSPKSHYVADKLFLWSEYWNHELLPNPT
REIGSGAAYWYALDDVRFSEKLNYDYIFLSQSRIS
SRLLSFAIEFALKNPQLQLLFSKHPDENIDLKNRI
IPDNLIISTESSIQGINESRVAVGVYSTSLFEALA
CGKQTFVVKYPGYEIMSNEIDSGLFFAVETPEEML
EKTSPNWVAVADIENQFFGQEKLEHHHHHH
(NmCSS)
SEQ ID NO: 3
MEKQNIAVILARQNSKGLPLKNLRKMNGISLLGHT
INAAISSKCFDRIIVSTDGGLIAEEAKNFGVEVVL
RPAELASDTASSISGVIHALETIGSNSGTVTLLQP
TSPLRTGAHIREAFSLFDEKIKGSVVSACPMEHHP
LKTLLQINNGEYAPMRHLSDLEQPRQQLPQAFRPN
GAIYINDTASLIANNCFFIAPTKLYIMSHQDSIDI
DTELDLQQAENILNHKES
(BLUSP)
SEQ ID NO: 4
MTEINDKAQLDIAAAGDTDAVTSDTPEETVNTPEV
DETFELSAAKMREHGMSETAINQFHHLYDVWRHEE
ASSWIREDDIEPLGHVPSFHDVYETINHDKAVDAF
AKTAFLKLNGGLGTSMGLDKAKSLLPVRRHKAKQM
RFIDIIIGQVLTARTRLNVELPLTFMNSFHTSADT
MKVLKHHRKFSQHDVPMEIIQHQEPKLVAATGEPV
SYPANPELEWCPPGHGDLFSTIWESGLLDVLEERG
FKYLFISNSDNLGARASRTLAQHFENTGAPFMAEV
AIRTKADRKGGHIVRDKATGRLILREMSQVHPDDK
EAAQDITKHPYFNTNSIWVRIDALKDKLAECDGVL
PLPVIRNKKTVNPTDPDSEQVIQLETAMGAAIGLF
NGSICVQVDRMRFLPVKTTNDLFIMRSDRFHLTDT
YEMEDGNYIFPNVELDPRYYKNIHDFDERFPYAVP
SLAAANSVSIQGDWTFGRDVMMFADAKLEDKGEPS
YVPNGEYVGPQGIEPDDWV
(SpGalK)
SEQ ID NO: 5
MAQHLTTEALRKDFLAVFGQEADQTFFSPGRINLI
GEHTDYNGGHVFPAAISLGTYGAARKRDDQVLRFY
SANFEDKGIIEVPLADLKFEKEHNWTNYPKGVLHF
LQEAGHVIDKGFDFYVYGNIPNGAGLSSSASLELL
TGVVAEHLFDLKLERLDLVKIGKQTENNFIGVNSG
IMDQFAIGMGADQRAIYLDTNTLEYDLVPLDLKDN
VVVIMNTNKRRELADSKYNERRAECEKAVEELQVS
LDIQTLGELDEWAVDQYSYLIKDENRLKRARHAVL
ENQRTLKAQVALQAGDLETFGRLMNASHVSLEHDY
EVTGLELDTLVHTAWAQEGVLGARMTGAGFGGCAI
ALVQKDTVEAFKEAVGKHYEEWGYAPSFYIAEVAG
GTRVLD
(PmPpA)
SEQ ID NO: 6
MGLETVPAGKALPDDIYWIEIPANSDPIKYEVDKE
SGALFVDRFMATAMFYPANYGYVNNTLSLDGDPVD
VLVPTPYPLQPGSVIRCRPVGVLKMTDEAGSDAKW
AVPHSKLTKEYDHIKDVNDLPALLKAQIQHFFESY
KALEAGKWVKVDGWEGVDAARQEILDSFERAKK
SEQ ID NO: 7
AGCTCATATGGCCGTTATTATTTTTGTGAATGGTA
TTCGTGCCG
SEQ ID NO: 8
AGCTAAGCTTTTACTTCTCTTGGCCGAAAAACTGG
TTTTCAATATCTGC
SEQ ID NO: 9
HHHHHH

What is claimed is:

1. A method for preparing a bacterial capsular saccharide product, the method comprising:
forming a reaction mixture containing one or more bacterial capsular polysaccharide synthases, a sugar acceptor, and one or more sugar donors; and
maintaining the reaction mixture under conditions sufficient to form the bacterial capsular saccharide product;
wherein the degree of polymerization of the bacterial capsular saccharide product ranges from 2 to about 200, and wherein the polydispersity index M_{w}/Mₙ of the bacterial capsular saccharide product ranges from 1 to about 1.5.

2. The method of claim 1, wherein the bacterial capsular saccharide product is a heteropolymer comprising disaccharide repeating units.

3. The method of claim 2, wherein forming the bacterial capsular saccharide product comprises glycosylating the sugar acceptor with monosaccharide residues of a first variety and monosaccharide residue of a second variety in alternating steps.

4. The method of claim 2, wherein forming the bacterial capsular saccharide product comprises glycosylating the sugar acceptor with alternating monosaccharide residues of a first variety and monosaccharide residues of a second variety in a single polymerization step.

5. The method of claim 1, the degree of polymerization of the bacterial capsular saccharide product ranges from 20 to about 200.

6. The method of claim 1, wherein the degree of polymerization of the bacterial capsulate saccharide product is greater than 50.

7. The method of claim 1, wherein the polydispersity index M_{w}/Mₙ of the bacterial capsular saccharide product ranges from 1.01 to about 1.15

8. The method of claim 1, wherein each bacterial capsular polysaccharide synthase is independently selected from N. meningitidis SiaD_{W} (NmSiaD_{W}), N. meningitidis SiaD_{Y} (NmSiaD_{Y}), a P. multocida heparosan synthase (PmHS1 and PmHS2), P. multocida hyaluronan synthase (PmHAS), S. pyogenes hyaluronan synthase (SpHAS), P. multocida chondroitin synthase (PmCS), E. coli K5 KfiA and KfiC, S. pneumoniae Type 3 capsular polysaccharide synthase (SpCps3 S), and S. pneumoniae Type 37 capsular polysaccharide synthase (SpCps37Tts).

9. The method of claim 8, wherein the reaction mixture comprises one bacterial capsular polysaccharide synthase, and wherein the bacterial capsular polysaccharide synthase is NmSiaD_{W}.

10. The method of claim 1, wherein the bacterial capsular saccharide product comprises galactose-sialic acid disaccharide repeating units.

11. The method of claim 1, wherein the galactose-sialic acid disaccharide repeating units are (-6Galα1-4Neu5Acα2).

12. The method of claim 10, wherein the reaction mixture comprises a galactose donor, a sialic acid donor, or a combination thereof.

13. The method of claim 12, wherein the galactose donor is UDP-Gal.

14. The method of claim 12, wherein the sialic acid donor is CMP-Neu5Ac.

15. The method of claim 10, wherein forming the bacterial capsular saccharide product comprises glycosylating the sugar acceptor with galactose residues and sialic acid residues in alternating steps.

16. The method of claim 10, wherein forming the bacterial capsular saccharide product comprises glycosylating the sugar acceptor with alternating galactose residues and sialic acid residues in a single polymerization step.

17. The method of claim 16, wherein the reaction mixture comprises UDP-Gal and CMP-Neu5Ac, and wherein the ratio (UDP-Gal+CMP-Neu5Ac):(sugar acceptor) ranges from about 1:1 to about 250:1.

18. The method of claim 17, wherein the ratio is about 100:1.

19. The method of claim 1, wherein the sugar acceptor comprises a sialic acid residue at its non-reducing end.

20. The method of claim 1, wherein the sugar acceptor comprises a galactose residue at its non-reducing end.

21. The method of claim 1, wherein the sugar acceptor comprises an oligosaccharide moiety Galα1-4Neu5Acα2(-6Galα1-4Neu5Acα2)ₙ-, or an oligosaccharide moiety Neu5Acα2(-6Galα1-4Neu5Acα2)ₘ-, wherein subscript n is 1, 2, 3, or 4 and subscript m is 1, 2, 3, 4, or 5.

22. The method of claim 1, wherein the acceptor comprises a purification handle.

23. The method of claim 1, wherein the reaction mixture further comprises a CMP-sialic acid synthetase, a nucleotide sugar pyrophosphorylase, a pyrophosphatase, a kinase, or a combination thereof.

24. The method of claim 23, wherein the CMP-sialic acid synthetase is NmCSS, wherein the nucleotide sugar pyrophosphorylase is BLUSP, wherein the pyrophosphatase is PmPpA, and wherein the kinase is SpGalK.

25. The method of claim 1, wherein the pH of the reaction mixture ranges from about 6 to about 9.

26. The method of claim 1, which is conducted in vitro.

27. A bacterial capsular saccharide product prepared according to the method of any one of claims 1-26.

28. A vaccine composition comprising a bacterial capsular saccharide product prepared according to the method of any one of claims 1-26 coupled to a carrier material.
